(19) United States
(12) Patent Application Publication
(10) Pub. No.: US 2004/0058015 A1
(43) Pub. Date: Mar. 25, 2004

(54) Compositions and methods for treating eye discomfort and eye disease

(76) Inventor: Yuanjin Tao, Fremont, CA (US)

Correspondence Address: Karen R. Zachow, Ph.D., Morrison & Foerster LLP, Suite 500, 3811 Valley Centre Drive, San Diego, CA, 92130-2332, US

(21) Appl. No.: 10/397,039

(22) Filed: Mar. 24, 2003

Related U.S. Application Data
(63) Continuation-in-part of application No. 09/872,403, filed on Jun. 1, 2001, now Pat. No. 6,537,581.
(60) Provisional application No. 60/208,783, filed on Jun. 1, 2000.

(51) Int. Cl. A61K 35/78; A61K 33/34; A61K 33/32; A61K 33/04

(52) U.S. Cl. 424/725; 424/757; 424/702; 424/750; 424/638; 424/642; 424/764; 424/732

(57) ABSTRACT

Compositions and methods for treating eye discomfort and eye disease are provided. The compositions comprise singly or in combination herbals, natural nutritional supplements, minerals and vitamins. Methods of making these compositions are also provided.

### CROSS-REFERENCE TO RELATED APPLICATIONS

The present application is a continuation-in-part application of copending U.S. patent application Ser. No. 09/872,403, filed Jun. 1, 2001, which in turn claims the benefit of U.S. Provisional Application Serial No. 60/208,783, filed Jun. 1, 2000, each of which is hereby incorporated in their entirety by reference.

### TECHNICAL FIELD

This invention relates to compositions and methods for treating eye disorders and eye diseases. More specifically, it relates to nutraceutical compositions for treating eye discomfort and disease symptoms, and methods for treating these symptoms with said compositions.

### BACKGROUND ART

Diabetic retinopathy, a complication of diabetes mellitus, is the leading cause of blindness in people ages 40 to 60 in the U.S. While diabetes can affect many parts of the eye, its most common manifestation takes the form of diabetic retinopathy and four-and-a-half million Americans are affected by diabetic retinopathy. Macular degeneration is an eye disease that can lead to blindness. Of the two forms of age-related macular degeneration, the dry form is more common and the wet form accounts for most of the case of legal blindness in macular degeneration. Glaucoma affects over 3 million Americans and is the second leading cause of blindness in the elderly in the United States. Cataract, generally associated with free radical damage to the cornea and most commonly related to aging, leads to clouding of the lens that can cause vision problems and often progresses to a point where surgery is required to improve vision. More than half of all Americans age 65 and older have a cataract.

One in five Americans suffers from discomfort of the eyes. One of the most common eye discomforts is due to dry eye condition. Dry eye conditions are characterized by ocular surface epithelium undergoing squamous metaplasia, loss of goblet cells, mucin deficiency and keratinization, leading to the clinical symptoms of dry eye syndrome. The advent of computer technology has accelerated the prevalence of vision disorders such as the dry eye syndromes. This is primarily due to the necessary use of video display terminals such as computer monitors.

The prevalence of dry eye discomfort in visual display terminal users and contact lens wearers is significantly higher than in non-visual display terminal users and non-contact lens wearers. It is suggested that dry eye is one of the most common ocular disorders encountered by physicians. Hijichi, T. et al., Graefes Arch. Clin. Exp. Ophthalmol. (1995), 233(9):555-8. 100061 The correlation between eye discomfort symptoms generally and work with visual display terminals has been clearly documented. The occurrence of eye discomfort increases as the extent of work involving visual display terminals increases. Symptoms include sensitivity to light, smarting and gritting feeling of the eyes, and eye redness. Bergqvist and Knave, Scand. J. Work Environ. Health (1994), 20(1):27-33. It is estimated that between 75 and 88 percent of patients who use video display terminals regularly are likely to be symptomatic for visual problems. Salibello, C. and Nilsen, E., J. Am. Optom. Assoc. (1995), 66(8):479-83. It has been found that, on the average, 14.25 percent of optometric patients present with symptoms primarily associated with the use of video display terminals, which translates into approximately 10 million cases annually in the U.S. population. Sheedy, J. E., J. Am. Optom. Assoc. (1992), 63(10):687-92. 100071 The use of video display terminals in the workplace is growing rapidly. It was estimated that more than 100 million workers in the United States and Canada would be using computers daily by the year 2000. Kirsner, R. S. and Federman, D. G., South Med. J. (1998), 91(1): 12-6. Therefore, it is reasonable to expect that there will be a significant increase in the prevalence of eye discomfort symptoms resulting from the use of computers.

Herbal remedies that are thought to be beneficial for improving visual functions generally are of unspecific efficacy for eye discomfort and disease. For example, a composition containing bilberry has been indicated to improve night vision acuity, field of vision and adaptation to light. U.S. Pat. No. 5,955,102. It is not disclosed whether this composition would be beneficial to individuals suffering from more specific (and more prevalent) eye discomfort conditions such as the dry eye syndrome.

A composition that is used for dry eye syndrome and supports the body's natural tear formation is known. (HydroEye™, U.S. Pat. No. 6,506,412, ScienceBased Health, Carson City, Nev.). The ingredients in this composition include vitamins A, C and B-6, magnesium sulfate, mucin complex, cod liver oil and black currant oil.

Multinutrient formulations with eye-specific nutrients are known. (MaculaRx® & MaculaRx® Plus, Science Based Health, Carson City, Nev.). The ingredients include vitamins A, B-6, B-12, C and E, magnesium, selenium, zinc, carotenoids, taurine, N-acetyl cysteine, lipoic acid, inositol, bilberry extract and ginkgo biloba extract.

The Ocuvite® PreserVision™ formulation (Bausch & Lomb, Inc.) is an antioxidant supplement with the antioxidant vitamins A, C and E and selected minerals. It's effect is being evaluated in the National Eye Institute's age-related eye study (AREDS) on progression of cataract and of age-related macular degeneration.

A multivitamin supplement with basic eye-specific nutrients is known. (Ocular Essentials™, ScienceBased Health, Carson City, NV). Its ingredients include Vitamins A, B1, B2, B3, B5, B6, B12, C, D3, E and K, biotin, folic acid, boron, calcium, chromium, copper, iodine, magnesium, manganese, molybdenum, potassium, sodium, selenium, zinc, carotenoids, zeaxanthin and cryptoxanthin, taurine, and bilberry extract.

A formulation for providing eye health and nutritional support is known. (OculaRx®, ScienceBased Health, Carson City, NV). Its ingredients include Vitamins A, B1, B2, B3, B5, B6, B12, C, D3 and E, biotin, folic acid, boron, calcium, chromium, copper, iodine, magnesium, manganses, molybdenum, phosphorus, selenium, zinc, bilberry extract, carotenoids, choline, ginkgo extract, grape seed extract, hesperidin bioflavonoid complex, lipoic acid, N-acetyl cysteine, rutin and taurine.

A composition for treating eye disorders is known. (Visianna Eye Refresher, Amino Foods Technologies, San Jose, Calif.). The ingredients in this formula include gochee berry extract (lycium barbarum L.), juhua (chrysanthemum morifolium Ramat), beta-carotene, vitamin E, ascorbic acid, riboflavin, zinc, copper, selenium and magnesium.

In view of the seriousness and prevalence of eye discomfort and eye disease symptoms, and the corresponding need to alleviate said symptoms without significant side effects, it would be advantageous to have compositions and methods that are efficacious and safe to address this need. The invention described and claimed in this specification presents such compositions and methods.

All references cited herein, including patent applications and publications, are incorporated by reference in their entirety.

### DISCLOSURE OF THE INVENTION

Compositions and methods for treating eye discomfort and eye disease are provided. The compositions comprise substances in quantities that are effective in relieving eye discomfort and eye disease. Methods of treating eye discomfort and eye disease comprising administration of the claimed compositions are also provided.

Accordingly, in one aspect, the invention provides a composition for treating eye discomfort comprising a substance selected from the group consisting of a member of the botanical group poria cocos, a member of the botanical group Cassia, a member of the botanical group Morus, a member of the botanical group Dioscorea, a member of the botanical group officinale and a member of the botanical group Rehmannia.

In another aspect, the invention provides a composition for treating eye discomfort comprising 2 substances selected from the group consisting of a member of the botanical group poria cocos, a member of the botanical group Cassia, a member of the botanical group Morus, a member of the botanical group Dioscorea, a member of the botanical group officinale and a member of the botanical group Rehmannia.

In another aspect, the invention provides a composition for treating eye discomfort comprising 3 substances selected from the group consisting of a member of the botanical group poria cocos, a member of the botanical group Cassia, a member of the botanical group Morus, a member of the botanical group Dioscorea, a member of the botanical group officinale and a member of the botanical group Rehmannia.

In another aspect, the invention provides a composition for treating eye discomfort comprising 4 substances selected from the group consisting of a member of the botanical group poria cocos, a member of the botanical group Cassia, a member of the botanical group Morus, a member of the botanical group Dioscorea, a member of the botanical group officinale and a member of the botanical group Rehmannia.

In another aspect, the invention provides a composition for treating eye discomfort comprising 5 substances selected from the group consisting of a member of the botanical group poria cocos, a member of the botanical group Cassia, a member of the botanical group Morus, a member of the botanical group Dioscorea, a member of the botanical group officinale and a member of the botanical group Rehmannia.

In another aspect, the invention provides a composition for treating eye discomfort comprising a member of the botanical group poria cocos, a member of the botanical group Cassia, a member of the botanical group Morus, a member of the botanical group Dioscorea, a member of the botanical group officinale and a member of the botanical group Rehmannia.

In one embodiment, the invention provides a composition selected from the group consisting of the compositions of the aspects of the invention described in the preceding paragraphs, further comprising a substance selected from the group consisting of a member of the botanical group Vaccinium, a member of the botanical group Salvia, a member of the botanical group Lycium, a member of the botanical group Chrysanthemum, a member of the botanical group Tribulus, beta-carotene, copper, a member of the botanical group Eriocaulon, magnesium, a member of the botanical group Paeonia suffruticosa, rice flour powder, selenium, vitamin B-2, vitamin C, vitamin E, a member of the botanical group Alisma and zinc. For example, a composition may comprise a member of the botanical group Rehmannia (such as shudihaung) and a member of the botanical group Lycium (gouqizhi). In another example, a composition may comprise a member of the botanical group Chrysanthemum (such as juhua) and a member of the botanical group Morus (such as sang ye).

In one embodiment, the invention provides a composition selected from the group consisting of the compositions of the aspects of the invention described in the preceding paragraphs, further comprising 2 substances selected from the group consisting of a member of the botanical group Vaccinium, a member of the botanical group Salvia, a member of the botanical group Lycium, a member of the botanical group Chrysanthemum, a member of the botanical group Tribulus, beta-carotene, copper, a member of the botanical group Eriocaulon, magnesium, a member of the botanical group Paeonia suffruticosa, rice flour powder, selenium, vitamin B-2, vitamin C, vitamin E, a member of the botanical group Alisma and zinc.

In one embodiment, the invention provides a composition selected from the group consisting of the compositions of the aspects of the invention described in the preceding paragraphs, further comprising 3 substances selected from the group consisting of a member of the botanical group Vaccinium, a member of the botanical group Salvia, a member of the botanical group Lycium, a member of the botanical group Chrysanthemum, a member of the botanical group Tribulus, beta-carotene, copper, a member of the botanical group Eriocaulon, magnesium, a member of the botanical group Paeonia suffruticosa, rice flour powder, selenium, vitamin B-2, vitamin C, vitamin E, a member of the botanical group Alisma and zinc.

In one embodiment, the invention provides a composition selected from the group consisting of the compositions of the aspects of the invention described in the preceding paragraphs, further comprising 4 substances selected from the group consisting of a member of the botanical group Vaccinium, a member of the botanical group Salvia, a member of the botanical group Lycium, a member of the botanical group Chrysanthemum, a member of the botanical group Tribulus, beta-carotene, copper, a member of the botanical group Eriocaulon, 0.5 magnesium, a member of the botanical group Paeonia suffruticosa, rice flour powder, selenium, vitamin B-2, vitamin C, vitamin E, a member of the botanical group Alisma and zinc.

In one embodiment, the invention provides a composition selected from the group consisting of the compositions of the aspects of the invention described in the preceding paragraphs, further comprising 5 substances selected from the group consisting of a member of the botanical group Vaccinium, a member of the botanical group Salvia, a member of the botanical group Lycium, a member of the botanical group Chrysanthemum, a member of the botanical group Tribulus, beta-carotene, copper, a member of the botanical group Eriocaulon, magnesium, a member of the botanical group Paeonia suffruticosa, rice flour powder, selenium, vitamin B-2, vitamin C, vitamin E, a member of the botanical group Alisma and zinc.

In one embodiment, the invention provides a composition selected from the group consisting of the compositions of the aspects of the invention described in the preceding paragraphs, further comprising 6 substances selected from the group consisting of a member of the botanical group Vaccinium, a member of the botanical group Salvia, a member of the botanical group Lycium, a member of the botanical group Chrysanthemum, a member of the botanical group Tribulus, beta-carotene, copper, a member of the botanical group Eriocaulon, magnesium, a member of the botanical group Paconia suffruticosa, rice flour powder, selenium, vitamin B-2, vitamin C, vitamin E, a member of the botanical group Alisma and zinc.

In one embodiment, the invention provides a composition selected from the group consisting of the compositions of the aspects of the invention described in the preceding paragraphs, further comprising 7 substances selected from the group consisting of a member of the botanical group Vaccinium, a member of the botanical group Salvia, a member of the botanical group Lycium, a member of the botanical group Chrysanthemum, a member of the botanical group Tribulus, beta-carotene, copper, a member of the botanical group Eriocaulon, magnesium, a member of the botanical group Paeonia suffruticosa, rice flour powder, selenium, vitamin B-2, vitamin C, vitamin E, a member of the botanical group Alisma and zinc.

In one embodiment, the invention provides a composition selected from the group consisting of the compositions of the aspects of the invention described in the preceding paragraphs, further comprising 8 substances selected from the group consisting of a member of the botanical group Vaccinium, a member of the botanical group Salvia, a member of the botanical group Lycium, a member of the botanical group Chrysanthemum, a member of the botanical group Tribulus, beta-carotene, copper, a member of the botanical group Eriocaulon, magnesium, a member of the botanical group Paeonia suffruticosa, rice flour powder, selenium, vitamin B-2, vitamin C, vitamin E, a member of the botanical group Alisma and zinc.

In one embodiment, the invention provides a composition selected from the group consisting of the compositions of the aspects of the invention described in the preceding paragraphs, further comprising 9 substances selected from the group consisting of a member of the botanical group Vaccinium, a member of the botanical group Salvia, a member of the botanical group Lycium, a member of the botanical group Chrysanthemum, a member of the botanical group Tribulus, beta-carotene, copper, a member of the botanical group Eriocaulon, magnesium, a member of the botanical group Paeonia suffruticosa, rice flour powder, selenium, vitamin B-2, vitamin C, vitamin E, a member of the botanical group Alisma and zinc.

In one embodiment, the invention provides a composition selected from the group consisting of the compositions of the aspects of the invention described in the preceding paragraphs, further comprising 10 substances selected from the group consisting of a member of the botanical group Vaccinium, a member of the botanical group Salvia, a member of the botanical group Lycium, a member of the botanical group Chrysanthemum, a member of the botanical group Tribulus, beta-carotene, copper, a member of the botanical group Eriocaulon, magnesium, a member of the botanical group Paeonia suffruticosa, rice flour powder, selenium, vitamin B-2, vitamin C, vitamin E, a member of the botanical group Alisma and zinc.

In one embodiment, the invention provides a composition selected from the group consisting of the compositions of the aspects of the invention described in the preceding paragraphs, further comprising 11 substances selected from the group consisting of a member of the botanical group Vaccinium, a member of the botanical group Salvia, a member of the botanical group Lycium, a member of the botanical group Chrysanthemum, a member of the botanical group Tribulus, beta-carotene, copper, a member of the botanical group Eriocaulon, magnesium, a member of the botanical group Paeonia suffruticosa, rice flour powder, selenium, vitamin B-2, vitamin C, vitamin E, a member of the botanical group Alisma and zinc.

In one embodiment, the invention provides a composition selected from the group consisting of the compositions of the aspects of the invention described in the preceding paragraphs, further comprising 12 substances selected from the group consisting of a member of the botanical group Vaccinium, a member of the botanical group Salvia, a member of the botanical group Lycium, a member of the botanical group Chrysanthemum, a member of the botanical group Tribulus, beta-carotene, copper, a member of the botanical group Eriocaulon, magnesium, a member of the botanical group Paeonia suffruticosa, rice flour powder, selenium, vitamin B-2, vitamin C, vitamin E, a member of the botanical group Alisma and zinc.

In one embodiment, the invention provides a composition selected from the group consisting of the compositions of the aspects of the invention described in the preceding paragraphs, further comprising 13 substances selected from the group consisting of a member of the botanical group Vaccinium, a member of the botanical group Salvia, a member of the botanical group Lycium, a member of the botanical group Chrysanthemum, a member of the botanical group Tribulus, beta-carotene, copper, a member of the botanical group Eriocaulon, magnesium, a member of the botanical group Paeonia suffruticosa, rice flour powder, selenium, vitamin B-2, vitamin C, vitamin E, a member of the botanical group Alisma and zinc.

In one embodiment, the invention provides a composition selected from the group consisting of the compositions of the aspects of the invention described in the preceding paragraphs, further comprising 14 substances selected from the group consisting of a member of the botanical group Vaccinium, a member of the botanical group Salvia, a member of the botanical group Lycium, a member of the botanical group Chrysanthemum, a member of the botanical group Tribulus, beta-carotene, copper, a member of the botanical group Eriocaulon, magnesium, a member of the botanical group Paeonia suffruticosa, rice flour powder, selenium, vitamin B-2, vitamin C, vitamin E, a member of the botanical group Alisma and zinc.

In one embodiment, the invention provides a composition selected from the group consisting of the compositions of the aspects of the invention described in the preceding paragraphs, further comprising 15 substances selected from the group consisting of a member of the botanical group Vaccinium, a member of the botanical group Salvia, a member of the botanical group Lycium, a member of the botanical group Chrysanthemum, a member of the botanical group Tribulus, beta-carotene, copper, a member of the botanical group Eriocaulon, magnesium, a member of the botanical group Paeonia suffruticosa, rice flour powder, selenium, vitamin B-2, vitamin C, vitamin E, a member of the botanical group Alisma and zinc.

In one embodiment, the invention provides a composition selected from the group consisting of the compositions of the aspects of the invention described in the preceding paragraphs, further comprising 16 substances selected from the group consisting of a member of the botanical group Vaccinium, a member of the botanical group Salvia, a member of the botanical group Lycium, a member of the botanical group Chrysanthemum, a member of the botanical group Tribulus, beta-carotene, copper, a member of the botanical group Eriocaulon, magnesium, a member of the botanical group Paeonia suffruticosa, rice flour powder, selenium, vitamin B-2, vitamin C, vitamin E, a member of the botanical group Alisma and zinc.

In one embodiment, the invention provides a composition selected from the group consisting of the compositions of the aspects of the invention described in the preceding paragraphs, further comprising a member of the botanical group Vaccinium, a member of the botanical group Salvia, a member of the botanical group Lycium, a member of the botanical group Chrysanthemum, a member of the botanical group Tribulus, beta-carotene, copper, a member of the botanical group Eriocaulon, magnesium, a member of the botanical group Paeonia suffruticosa, rice flour powder, selenium, vitamin B-2, vitamin C, vitamin E, a member of the botanical group Alisma and zinc.

In various embodiments of the compositions of the invention described in the preceding and following paragraphs, the member of the botanical group poria cocos may be fuling, the member of the botanical group Cassia may be jueminzi, the member of the botanical group Morus may be sang ye, the member of the botanical group Dioscorea may be shanyao, the member of the botanical group officinale may be shanzhuyu, the member of the botanical group Rehmannia may be shudihaung, the member of the botanical group Vaccinium may be bilberry, the member of the botanical group Salvia may be danshen, the member of the botanical group Lycium may be gouqizhi, the member of the botanical group Chrysanthemum may be juhua, the member of the botanical group Tribulus may be baijili, the member of the botanical group Eriocaulon may be gujincao, the member of the botanical group Paconia suffruticosa may be mudanpi, and/or the member of the botanical group Alisma may be zexie. Accordingly, in one embodiment, the invention provides a composition for treating eye discomfort comprising fuling, jueminzi, sang ye, shanyao, shanzhuyu, shudihaung, bilberry, danshen, gouqizhi, juhua, baijili, beta-carotene, copper, gujincao, magnesium, mudanpi, rice flour powder, selenium, vitamin B-2, vitamin C, vitamin E, zexie and zinc.

Accordingly, in one aspect, the invention provides a composition for treating eye discomfort comprising a substance selected from the group consisting of fuling, jueminzi, sang ye, shanyao, shanzhuyu and shudihaung.

In another aspect, the invention provides a composition for treating eye discomfort comprising 2 substances selected from the group consisting of fuling, jueminzi, sang ye, shanyao, shanzhuyu and shudihaung.

In another aspect, the invention provides a composition for treating eye discomfort comprising 3 substances selected from the group consisting of fuling, jueminzi, sang ye, shanyao, shanzhuyu and shudihaung.

In another aspect, the invention provides a composition for treating eye discomfort comprising 4 substances selected from the group consisting of fuling, jueminzi, sang ye, shanyao, shanzhuyu and shudihaung.

In another aspect, the invention provides a composition for treating eye discomfort comprising 5 substances selected from the group consisting of fuling, jueminzi, sang ye, shanyao, shanzhuyu and shudihaung.

In yet another aspect, the invention provides a composition for treating eye discomfort comprising fuling, jueminzi, sang ye, shanyao, shanzhuyu and shudihaung.

In one embodiment, the invention provides a composition selected from the group consisting of the compositions of the aspects of the invention described in the preceding paragraphs, further comprising a substance selected from the group consisting of bilberry, danshen, gouqizhi, juhua, baijili, beta-carotene, copper, gujincao, magnesium, mudanpi, rice flour powder, selenium, vitamin B-2, vitamin C, vitamin E, zexie and zinc.

In another embodiment, the invention provides a composition selected from the group consisting of the compositions of the aspects of the invention described in the preceding paragraphs, further comprising 2 substances selected from the group consisting of bilberry, danshen, gouqizhi, juhua, baijili, beta-carotene, copper, gujincao, magnesium, mudanpi, rice flour powder, selenium, vitamin B-2, vitamin C, vitamin E, zexie and zinc.

In another embodiment, the invention provides a composition selected from the group consisting of the compositions of the aspects of the invention described in the preceding paragraphs, further comprising 3 substances selected from the group consisting of bilberry, danshen, gouqizhi, juhua, baijili, beta-carotene, copper, gujincao, magnesium, mudanpi, rice flour powder, selenium, vitamin B-2, vitamin C, vitamin E, zexie and zinc.

In another embodiment, the invention provides a composition selected from the group consisting of the compositions of the aspects of the invention described in the preceding paragraphs, further comprising 4 substances selected from the group consisting of bilberry, danshen, gouqizhi, juhua, baijili, beta-carotene, copper, gujincao, magnesium, mudanpi, rice flour powder, selenium, vitamin B-2, vitamin C, vitamin E, zexie and zinc.

In another embodiment, the invention provides a composition selected from the group consisting of the compositions of the aspects of the invention described in the preceding paragraphs, further comprising 5 substances selected from the group consisting of bilberry, danshen, gouqizhi, juhua, baijili, beta-carotene, copper, gujincao, magnesium, mudanpi, rice flour powder, selenium, vitamin B-2, vitamin C, vitamin E, zexie and zinc.

In another embodiment, the invention provides a composition selected from the group consisting of the compositions of the aspects of the invention described in the preceding paragraphs, further comprising 6 substances selected from the group consisting of bilberry, danshen, gouqizhi, juhua, baijili, beta-carotene, copper, gujincao, magnesium, mudanpi, rice flour powder, selenium, vitamin B-2, vitamin C, vitamin E, zexie and zinc.

In another embodiment, the invention provides a composition selected from the group consisting of the compositions of the aspects of the invention described in the preceding paragraphs, further comprising 7 substances selected from the group consisting of bilberry, danshen, gouqizhi, juhua, baijili, beta-carotene, copper, gujincao, magnesium, mudanpi, rice flour powder, selenium, vitamin B-2, vitamin C, vitamin E, zexie and zinc.

In another embodiment, the invention provides a composition selected from the group consisting of the compositions of the aspects of the invention described in the preceding paragraphs, further comprising 8 substances selected from the group consisting of bilberry, danshen, gouqizhi, juhua, baijili, beta-carotene, copper, gujincao, magnesium, mudanpi, rice flour powder, selenium, vitamin B-2, vitamin C, vitamin E, zexie and zinc.

In another embodiment, the invention provides a composition selected from the group consisting of the compositions of the aspects of the invention described in the preceding paragraphs, further comprising 9 substances selected from the group consisting of bilberry, danshen, gouqizhi, juhua, baijili, beta-carotene, copper, gujincao, magnesium, mudanpi, rice flour powder, selenium, vitamin B-2, vitamin C, vitamin E, zexie and zinc.

In another embodiment, the invention provides a composition selected from the group consisting of the compositions of the aspects of the invention described in the preceding paragraphs, further comprising 10 substances selected from the group consisting of bilberry, danshen, gouqizhi, juhua, baijili, beta-carotene, copper, gujincao, magnesium, mudanpi, rice flour powder, selenium, vitamin B-2, vitamin C, vitamin E, zexie and zinc.

In another embodiment, the invention provides a composition selected from the group consisting of the compositions of the aspects of the invention described in the preceding paragraphs, further comprising 11 substances selected from the group consisting of bilberry, danshen, gouqizhi, juhua, baijili, beta-carotene, copper, gujincao, magnesium, mudanpi, rice flour powder, selenium, vitamin B-2, vitamin C, vitamin E, zexie and zinc.

In another embodiment, the invention provides a composition selected from the group consisting of the compositions of the aspects of the invention described in the preceding paragraphs, further comprising 12 substances selected from the group consisting of bilberry, danshen, gouqizhi, juhua, baijili, beta-carotene, copper, gujincao, magnesium, mudanpi, rice flour powder, selenium, vitamin B-2, vitamin C, vitamin E, zexie and zinc.

In another embodiment, the invention provides a composition selected from the group consisting of the compositions of the aspects of the invention described in the preceding paragraphs, further comprising 13 substances selected from the group consisting of bilberry, danshen, gouqizhi, juhua, baijili, beta-carotene, copper, gujincao, magnesium, mudanpi, rice flour powder, selenium, vitamin B-2, vitamin C, vitamin E, zexie and zinc.

In another embodiment, the invention provides a composition selected from the group consisting of the compositions of the aspects of the invention described in the preceding paragraphs, further comprising 14 substances selected from the group consisting of bilberry, danshen, gouqizhi, juhua, baijili, beta-carotene, copper, gujincao, magnesium, mudanpi, rice flour powder, selenium, vitamin B-2, vitamin C, vitamin E, zexie and zinc.

In another embodiment, the invention provides a composition selected from the group consisting of the compositions of the aspects of the invention described in the preceding paragraphs, further comprising 15 substances selected from the group consisting of bilberry, danshen, gouqizhi, juhua, baijili, beta-carotene, copper, gujincao, magnesium, mudanpi, rice flour powder, selenium, vitamin B-2, vitamin C, vitamin E, zexie and zinc.

In another embodiment, the invention provides a composition selected from the group consisting of the compositions of the aspects of the invention described in the preceding paragraphs, further comprising 16 substances selected from the group consisting of bilberry, danshen, gouqizhi, juhua, baijili, beta-carotene, copper, gujincao, magnesium, mudanpi, rice flour powder, selenium, vitamin B-2, vitamin C, vitamin E, zexie and zinc.

In yet another embodiment, the invention provides a composition selected from the group consisting of the compositions of the aspects of the invention described in the preceding paragraphs, further comprising bilberry, danshen, gouqizhi, juhua, baijili, beta-carotene, copper, gujincao, magnesium, mudanpi, rice flour powder, selenium, vitamin B-2, vitamin C, vitamin E, zexie and zinc.

In another aspect, the invention provides a composition for treating eye disease comprising a member of the botanical group Poria cocos, a member of the botanical group Salvia, a member of the botanical group Leonurus, a member of the botanical group Broussonetia, a member of the botanical group Lycium, a member of the botanical group Panax, a member of the botanical group Chrysanthemum, a member of the botanical group Paeonia suffruticosa, a member of the botanical group Dioscorea, a member of the botanical group officinale, a member of the botanical group Rehmannia, a member of the botanical group Alisma, a member of the botanical group Glycyrrhiza and a member of the botanical group Corydalis. In one embodiment, the invention provides this composition for treating eye disease further comprising at least one substance selected from the group consisting of beta-carotene, copper, magnesium, rice flour powder, selenium, vitamin B-2, vitamin C, vitamin E, L-carnitine and zinc.

In another embodiment, the invention provides a composition for treating eye disease comprising danshen, chongweizi, fuling, zhushizi, gouqizhi, shanqi, juhua, mudanpi, shanyao, shanzhuyu, shudihaung, zexie, gancao and yanhusuo.

In another embodiment, the invention provides a composition for treating eye disease comprising danshen, chongweizi, fuling, zhushizi, gouqizhi, shanqi, juhua, mudanpi, shanyao, shanzhuyu, shudihaung, zexie, gancao, yanhusuo, beta-carotene, copper, magnesium, rice flour powder, selenium, vitamin B-2, vitamin C, vitamin E, L-carnitine and zinc.

In another aspect, the invention provides a composition for treating eye disease comprising a member of the botanical group Poria cocos, a member of the botanical group Salvia, a member of the botanical group Puerariae, a member of the botanical group Astragalus, a member of the botanical group Lycium, a member of the botanical group Panax, a member of the botanical group Chrysanthemum, a member of the botanical group Paeonia suffruticosa, a member of the botanical group Dioscorea, a member of the botanical group Paeonia, a member of the botanical group officinale, a member of the botanical group Rehmannia, a member of the botanical group Alisma, a member of the botanical group Glycyrrhiza and a member of the botanical group Corydalis. In one embodiment, the invention provides this composition for treating eye disease further comprising at least one substance selected from the group consisting of beta-carotene, copper, magnesium, rice flour powder, selenium, vitamin B-2, vitamin C, vitamin E, L-carnitine and zinc.

In another embodiment, the invention provides a composition for treating eye disease comprising danshen, gegen, fuling, baishaoyao, gouqizhi, shanqi, juhua, yanhusuo, mudanpi, shanyao, shanzhuyu, shudihaung, zexie, gancao, and huangqi.

In another embodiment, the invention provides a composition for treating eye disease comprising danshen, gegen, fuling, baishaoyao, gouqizhi, shanqi, juhua, yanhusuo, mudanpi, shanyao, shanzhuyu, shudihaung, zexie, gancao, huangqi, beta-carotene, copper, magnesium, rice flour powder, selenium, vitamin B-2, vitamin C, vitamin E, L-carnitine and zinc.

In one aspect, the invention provides a method of treating eye discomfort comprising administering to the individual an effective amount of a composition selected from the group consisting of the compositions of the aspects and embodiments of the invention described in the preceding paragraphs, whereby eye discomfort is treated. In some embodiments, said eye discomfort is a condition selected from the group consisting of dry eye syndrome, itchy and red eye condition, eye fatigue, and eye sandiness and soreness.

In one aspect, the invention provides a method of treating eye disease comprising administering to the individual an effective amount of a composition selected from the group consisting of the compositions of the aspects and embodiments of the invention described in the preceding paragraphs, whereby eye disease is treated. In some embodiments, said eye disease is selected from the group consisting of macular degeneration (including age-related macular degeneration), diabetic retinopathy, cataract, glaucoma and conjunctiva.

In another aspect, the invention provides a method of making a composition for treating eye discomfort, said method comprising combining at least two (preferably 2, 3, 4, 5 or 6) substances (preferably in an effective amount) selected from the group consisting of a member of the botanical group poria cocos, a member of the botanical group Cassia, a member of the botanical group Morus, a member of the botanical group Dioscorea, a member of the botanical group officinale and a member of the botanical group Rehmannia. In some embodiments of these methods, said at least two substances are combined with at least one (preferably 1, 2, 3, 4, 5, 6, 7, 8, 9, 10, 11, 12, 13, 14, 15, 16, 17) substance (preferably an effective amount) selected from the group consisting of a member of the botanical group Vaccinium, a member of the botanical group Salvia, a member of the botanical group Lycium, a member of the botanical group Chrysanthemum, a member of the botanical group Tribulus, beta-carotene, copper, a member of the botanical group Eriocaulon, magnesium, a member of the botanical group Paeonia suffruticosa, rice flour powder, selenium, vitamin B-2, vitamin C, vitamin E, a member of the botanical group Alisma and zinc. In some embodiments, said combining is by mixing (such as by stirring, agitation or vibration). In some embodiments, the substances are packaged in the form of capsules, preferably in size “0”, “00”, “000”, “1”, “2”, “3” or “4.” In yet other embodiments, the substances are combined in powder form, preferably to at least 30%, 60%, or 90% mixture consistency, or to homogeneity.

In another aspect, the invention provides a method of making a composition for treating eye disease, said method comprising combining at least three (preferably 3, 4, 5, 6, 7, 8, 9, 10, 11, 12, 13 or 14) substances (preferably in an effective amount) selected from the group consisting of a member of the botanical group Poria cocos, a member of the botanical group Salvia, a member of the botanical group Leonurus, a member of the botanical group Broussonetia, a member of the botanical group Lycium, a member of the botanical group Panax, a member of the botanical group Chrysanthemum, a member of the botanical group Paeonia suffruticosa, a member of the botanical group Dioscorea, a member of the botanical group officinale, a member of the botanical group Rehmannia, a member of the botanical group Alisma, a member of the botanical group Glycyrrhiza and a member of the botanical group Corydalis. In some embodiments of these methods, said at least three substances are combined with at least one (preferably 1, 2, 3, 4, 5, 6, 7, 8, 9 or 10) substance (preferably an effective amount) selected from the group consisting of a beta-carotene, copper, magnesium, rice flour powder, selenium, vitamin B-2, vitamin C, vitamin E, L-carnitine and zinc. In some embodiments, said combining is by mixing (such as by stirring, agitation or vibration). In some embodiments, the substances are packaged in the form of capsules, preferably in size “0”, “00”, “000”, “1”, “2”, “3” or “4.” In yet other embodiments, the substances are combined in powder form, preferably to at least 30%, 60%, or 90% mixture consistency, or to homogeneity.

In another aspect, the invention provides a method of making a composition for treating eye disease, said method comprising combining at least three (preferably 3, 4, 5, 6, 7, 8, 9, 10, 11, 12, 13, 14 or 15) substances (preferably in an effective amount) selected from the group consisting of a member of the botanical group Poria cocos, a member of the botanical group Salvia, a member of the botanical group Puerariae, a member of the botanical group Astragalus, a member of the botanical group Lycium, a member of the botanical group Panax, a member of the botanical group Chrysanthemum, a member of the botanical group Paeonia suffruticosa, a member of the botanical group Dioscorea, a member of the botanical group Paeonia, a member of the botanical group officinale, a member of the botanical group Rehmannia, a member of the botanical group Alisma, a member of the botanical group Glycyrrhiza and a member of the botanical group Corydalis. In some embodiments of these methods, said at least three substances are combined with at least one (preferably 1, 2, 3, 4, 5, 6, 7, 8, 9 or 10) substance (preferably an effective amount) selected from the group consisting of a beta-carotene, copper, magnesium, rice flour powder, selenium, vitamin B-2, vitamin C, vitamin E, L-carnitine and zinc. In some embodiments, said combining is by mixing (such as by stirring, agitation or vibration). In some embodiments, the substances are packaged in the form of capsules, preferably in size “0”, “00”, “000”, “1”, “2”, “3” or “4.” In yet other embodiments, the substances are combined in powder form, preferably to at least 30%, 60%, or 90% mixture consistency, or to homogeneity.

### MODES FOR CARRYING OUT THE INVENTION

The present invention discloses compositions comprising single or multiple substances that are effective and safe for treating symptoms of eye discomfort and eye disease. The invention further provides methods of treating eye discomfort and eye disease using the disclosed compositions. Methods of making these compositions are also provided.

Definitions

The term “treating,” “treatment,” and variations thereof, as used in this specification, refers to an approach for obtaining beneficial or desired physiological results, which may be established clinically. For purposes of this invention, beneficial or desired clinical results include, but are not limited to, alleviation of symptoms, diminishment of extent of disease, stabilized (i.e., not worsening) condition, delay or slowing of progression or worsening of condition/symptoms, amelioration or palliation of the condition or symptoms, and remission (whether partial or total), whether detectable or undetectable. The term “palliation”, and variations thereof, as used herein, means that the extent and/or undesirable manifestations of a physiological condition or symptom are lessened and/or time course of the progression is slowed or lengthened, as compared to not administering compositions of the present invention.

A “treatment effect” or “therapeutic effect” is manifested if there is a change in the condition being treated, as measured by the criteria constituting the definition of the terms “treating” and “treatment.” There is a “change” in the condition being treated if there is at least 10% improvement, preferably at least 25%, more preferably at least 50%, even more preferably at least 75%, and most preferably at least 100%. The change can be based on improvements in the severity of the treated condition in an individual, or on a difference in the frequency of improved conditions in populations of individuals with and without treatment with the compositions of the present invention.

An “effective amount” is an amount of a composition or substance(s) sufficient to effect beneficial or desired results in the treatment of eye discomfort after one or more administrations of that amount. An effective amount can be administered in one administration, or through multiple administrations of an amount that total an effective amount, preferably within a 24-hour period. It can be determined using standard clinical procedures for determining appropriate amounts and timing of administration. It is understood that the “effective amount” can be the result of empirical and/or individualized (case-by-case) determination on the part of the treating health care professional and/or individual.

“Eye discomfort,” as used herein, refers to symptoms involving the eye that prevent the normal functioning or use of the eye. These symptoms can be those presently indicated as manifestations or any condition that an individual finds to be non-conducive to the optimal or normal use of the eye. Such symptoms include, but are not limited to, burning, irritation, dryness, redness, excess tearing, blurred vision, difficulty with visual focusing, eye fatigue, soreness, eye strain and/or watery eye, including such symptoms that are due to contact lens use.

“Eye disease,” as used herein, refers to symptoms of a pathological condition involving the eye and/or associated structures. These symptoms can be the result of infection of and/or degenerative processes in the eye, cells or substances of the eye and/or cells or structures necessary to support normal functioning or use of the eye. Such diseases include, but are not limited to, macular degeneration, including age-related macular degeneration (ARMD), vascular retinopathies, including diabetic retinopathy, hypertensive retinopathy, central retinal artery occlusion and central retinal vein occlusion, papillitis, retrobulbar neuritis, optic atrophy, cataract, glaucoma and conjunctiva. In some diseases, decreases in microcirculation can lead to angiogenesis and the formation of fragile small blood vessels which can cause bleeding, scarring, may necessitate laser surgery and may lead to eventual blindness. Symptoms associated with such diseases include, but are not limited to, diminished and/or blurred vision, and/or pain, associated, for example, with ocular pressure. Symptoms associated with macular degeneration, including ARMD, are known in the art and include, but are not limited to, straight lines appearing distorted and, in some cases, the center of vision appearing more distorted than the rest of the scene; a dark, blurry area or “white-out” appears in the center of vision; color perception changes or diminishes; declining vision noticed by patient or by an ophthalmologist during a routine eye exam; formation of new blood vessels and/or exudates, or “drusen,” from blood vessels in and/or under the macular. Symptoms associated with vascular retinopathies, including diabetic retinopathy, are known in the art and include, but are not limited to, blurred vision; hazy vision; floaters (moving spots) or other patterns in the visual field; change (increase or decrease) in diameter in retina blood vessel(s); insufficient blood supply to areas of the retina; growth of blood vessels near the surface of the retina; blood leaking from vessels into the vitreous; retinal detachment; retinal scar tissue. Symptoms associated with glaucoma are known in the art and include, but are not limited to, blurred vision; gradual reduction in and loss of peripheral (side) vision; high IOP (intraocular pressure) level, for example, as measured by a tonometer; changes in the optic nerve, for example, as determined with an opthalmoscope.

Age-related macular degeneration (ARMD) occurs in two forms, “wet” and “dry.” may occur in younger and in older patients. The majority of patients, in some estimates approximately 90%, have the “dry” form, which involves thinning of the macular tissues and disturbances in its pigmentation. The minority, approximately 10%, of patients have the “wet” form, which can involve bleeding within and/or beneath the retina, opaque deposits, pooling of proteinacious material beneath the sheath, eventually scar tissue and/or detachment of the retina. The “wet” form accounts for ninety percent of all cases of legal blindness in macular degeneration patients. Different forms of macular degeneration may occur in younger patients. These non-age related cases may be linked to heredity, diabetes, nutritional deficits, head injury, infection, or other factors.

“Individual,” as used herein, refers to a vertebrate, preferably a mammal, more preferably a human.

A “botanical group,” as used herein, refers to a group of botanical entities that are capable of providing similar physiological effect(s) in the compositions of the invention. These botanical entities may or may not belong to the same botanical classification (such as genus, family).

“Fuling,” as used herein, refers to extracts of fuling. It is also known as sclerotium poriae cocos and poria cocos (Schw.) wolf. A member of the botanical group poria cocos is a substance that is capable of providing a similar physiological effect(s) as that provided by fuling in the compositions of the invention, and is preferably selected from a group comprising Poria cocos(schw.)wolf.; Sclerotium poriae cocos.

“Jueminzi,” as used herein, refers to extracts of jueminzi. It is also known as semen cassiae and cossia obtusifolia L. A member of the botanical group Cassia is a substance that is capable of providing a similar physiological effect(s) as that provided by jueminzi in the compositions of the invention, and is preferably selected from a group comprising Cassia obtusifolia L.; Cassia tora L.; Semen cassiae; Senna tora; Cassia tora auct. non; Emelista tora; Emelista tora sensu; Cassia tora; Cassia obtusifolia; Cassia tora auct. Non; Emelista tora; Emelista tora sensu; Semen obtusifolia.

“Sang ye,” as used herein, refers to extracts of sang ye. It is also known as folium mori albae and morus alba L. A member of the botanical group Morus is a substance that is capable of providing a similar physiological effect(s) as that provided by sang ye in the compositions of the invention, and is preferably selected from a group comprising Morus Folium mori albae; Morus alba L.; Morus australis; Morus australis Poir; Morus mongolica Schneid; Morus cathayana Hemsl.; Morus laevigata wall; Morus acidosa(Griff.); Morus alba stylosa((Ser.)Bur.); Morus indica(non L.); Morus stylosa(Ser.); Morus alba; Morus alba f. tatarica; Mortus alba var. constantinopolitana; Morus alba var. multicaulis; Morus alba var. stylosa; Morus alba var. tatarica; Morus constantinopolitana; Morus multicaulis; Morus tatarica.

“Shanyao,” as used herein, refers to extracts of shanyao. It is also known as radix dioscoreae oppositae and dioscorea opposita Thunb. A member of the botanical group Dioscorea is a substance that is capable of providing a similar physiological effect(s) as that provided by shanyao in the compositions of the invention, and is preferably selected from a group comprising Radix dioscoreae oppositae; Dioscorea batatas; Dioscorea opposita Thunb; Dioscorea batatas Decne.; Dioscorea japonica Thunb.; Dioscorea divaricata; Dioscorea opposita (Thunb.).

“Shanzhuyu,” as used herein, refers to extracts of shanzhuyu. It is also known as fructus corni officinalis and cornus officinalis sieb. A member of the botanical group officinale is a substance that is capable of providing a similar physiological effect(s) as that provided by shanzhuyu in the compositions of the invention, and is preferably selected from a group comprising Fructus corni officinalis; Cornus officalis Sieb.et.Zucc.; Macrocarpium officinale (Sieb. et Zucc) Nakai.

“Shudihaung,” as used herein, refers to extracts of shudihaung. It is also known as radix rehmanniae glutinosae conquitae and rehmannia glutinosa (Gaertn) Libosch. A member of the botanical group Rehmannia is a substance that is capable of providing a similar physiological effect(s) as that provided by shudihaung in the compositions of the invention, and is preferably selected from a group comprising Radix rehmanniae; Radix rehmanniae glutinosae conquitae; Rehmannia glutinosa(Gaertn)Libosch., f. hueichinggensis (Chao et Schih) Hsiao; Rehmannia glutinosa(Gaertn)Libosch; Rehmannia glutinosa(Gaertn)Libosch.var. lutea Makino, f. purpurea Makino; Rehmannia chinensis; Digitalis glutinosa.

“Bilberry,” as used herein, refers to extracts of bilberry. It is also known as vaccinium myrtillus. A member of the botanical group Vaccinium is a substance that is capable of providing a similar physiological effect(s) as that provided by bilberry in the compositions of the invention, and is preferably selected from a group comprising Vaccinium myrtillus; Vaccinium frondosum

“Danshen,” as used herein, refers to extracts of danshen. It is also known as radix salviae miltiorrhizae and salvia miltiorrhiza Bge. A member of the botanical group salvia is a substance that is capable of providing a similar physiological effect(s) as that provided by danshen in the compositions of the invention, and is preferably selected from a group comprising Radix salviae miltiorrhizae; Salvia miltiorrhiza; Salviae multiorrhiza Bge; Salvia bowleyana Dunn; Salvia przewalskii Maxim.; Salvia przewalskii Maxim.var.mandarinorum(Diels)Stib.; Salvia yunnannensis C. H. Wright; Salvia kiaometiensis Levl., f.pubescensis; Salvia miltiorrhiza Bunge var.alba C. Y. Wu et H. W. Li. mss.; Salvia digitaloides Diels; Salvia trijuga Diels; Salvia plectranthoides Griff.

“Gouqizhi,” as used herein, refers to extracts of gouqizhi. It is also known as fructus lycii and lycium barbarum L. A member of the botanical group Lycium is a substance that is capable of providing a similar physiological effect(s) as that provided by gouqizhi in the compositions of the invention, and is preferably selected from a group comprising Fructus lycii; Lycium barbarum L.; Lycium chinense Mill.; Lycium turcomanicum Turcz; Lycium potaninii Pojank; Lycium dasystemum Pojank; Lycium europaeum (non L.); Lycium halimifolium (Mill.); Lycium lanceolatum (Veillard.); Lycium megistocarpum (Dun.); Lycium ovatum.; Lycium subglobosum.; Lycium trewianum. L. vu; Lycium europeum; Lycium halamifolium; Lycium halmifolium; Lycium vulgare.

“Juhua,” as used herein, refers to extracts of juhua. It is also known as Flos chrysanthemi morifolii and chrysanthemum morifolium Ramat. A member of the botanical group Chrysanthemum is a substance that is capable of providing a similar physiological effect(s) as that provided by juhua in the compositions of the invention, and is preferably selected from a group comprising Flos chrysanthemi morifolii; Chrysanthemum morifolium Ramat.; Chrysanthemum sinense; Chrysanthemum×morifolium (Ramat.); Chrysanthemum indicum; Chrysanthemum×morifolium; Chrysanthemum indicum×sinense; Chrysanthemum morifolium; Chrysanthemum stipulaceum; Dendranthema×morifolium (Tzvelev.); Dendranthema indicum; Dendranthema×grandiflorum; Gnaphalium indicum; Gnaphalium indicum auct. Non.

“Baijili,” as used herein, refers to extracts of baijili. It is also known as fructus tribuli terrestris and tribulus terrestris L. A member of the botanical group Tribulus is a substance that is capable of providing a similar physiological effect(s) as that provided by baijili in the compositions of the invention, and is preferably selected from a group comprising Fructus tribuli terrestris; Tribulus terrestris L; Atriplex centralasiatica; Atriplex sibirica L; Pedalium murex; Tribulus terrestes; Tribulus terrestrus.

“Beta-carotene,” as used herein, refers to the form(s) of Vitamin A known to persons of skill in the art as such.

“Copper,” as used herein, refers to the form(s) of the mineral known to persons of skill in the art to be therapeutically effective in the body of the individual. It is preferably provided as copper gluconate or cupric oxide.

“Gujincao,” as used herein, refers to extracts of gujincao. It is also known as eriocaulon sieboldtianum S. A member of the botanical group Eriocaulon is a substance that is capable of providing a similar physiological effect(s) as that provided by gujincao in the compositions of the invention, and is preferably selected from a group comprising Eriocaulon buergerianum Koem.; Eriocaulon sieboldtianum Sieh.et Zucc. Ex Steud; Eriocaulon sieboldtianum S. et Z.; Eriocaulon sieboldianum; Eriocaulon saxangulara L.; Eriocaulon wallichianum Mart.

“Magnesium,” as used herein, refers to the form(s) of the mineral known to persons of skill in the art to be therapeutically effective in the body of the individual. It is preferably provided as magnesium stearate NF.

“Mudanpi,” as used herein, refers to extracts of mudanpi. It is also known as cortex moutan radicis and paeonia suffruticosa Andr. A member of the botanical group Paeonia suffruticosa is a substance that is capable of providing a similar physiological effect(s) as that provided by mudanpi in the compositions of the invention, and is preferably selected from a group comprising Cortex moutan radicis; Paeonia suffruticosa; Paeonia suffruticosa Andr.; Paeonia suffruticosa Andr. var. spontanea Rehd.; Paeonia suffruticosa Andr. Var. papaveracea (Andr.) Kerner; Paeonia papaveracea Andr.; Paeonia lutea Franch.; Paeonia szechuanica Fang; Paeonia moutan. Sims.; Paeonia moutan; Paeonia arborea.

“Shanqi,” as used herein, refers to extracts of shanqi. It is also known as Radix Notoginsheng and Panax notoginsheng (Burk.) F. H. Chen. A member of the botanical group Panax is a substance that is capable of providing a similar physiological effect(s) as that provided by shanqi in the compositions of the invention, and is preferably selected from a group comprising Radix notoginsheng; Panax notoginsheng (Burk.) F. H. Chen.; Panax notoginseng (Burk.) F. H. Chen (Shanqi); Panax pseudo-ginseng Wall.var.notoginseng (Burk) Hoo et Tseng; Panax ginseng C. A. Mey (P. schinseng Nees); Panax guinquefolium L.; Radix ginseng; Panax ginseng C. A. Mey; Panax schin-seng Nees; Panax schinseng.; Panax schinseng; Panax pseudoginseng; Panax pseudoginseng japonicus (C. A. Mey.) Hoo. & Tseng.; Panax repens. Max.; Panax pseudoginseng japonicus; Panax pseudoginseng ssp. Japonicus; Panax pseudoginseng subsp.; Panax japonicus; Panax pseudoginseng var. japonicus; Panax pseudo-ginseng; Panax ginseng; Panax schinseng Acanthopanax sentocosus (Rupr. et Maxim.); Eleutherococus senticosus (Rupr. et Maxim.).; Eleutherococus senticosus; Acanthopanax senticosus; Acanthopanax seticosus Harms; Acantherococus senticocus (Rupr. et Maxim) Harms.; Fleutherococus senticocus (Rupr. Et Maxim) Maxim.; Echinopanax elatus Nakai; Hedera senticosa.

“Gancao,” as used herein, refers to extracts of gancao. It is also known as Radix glycyrrhizae uralensis, Glycyrrhiza uralensis Fischer or licorice root. A member of the botanical group glycyrrhiza is a substance that is capable of providing a similar physiological effect(s) as that provided by gancao in the compositions of the invention, and is preferably selected from a group comprising Radix glycyrrhizae uralensis; Glycyrrhiza uralensis Fischer; Glycyrrhiza uralensis Fisch (Gan cao); Glycyrrhiza glabra L.; Glycyrrhizainflata Batal.; Glycyrrihiza korshiskyi G. Hrig.; Glycyrrhiza aspera Pall; Glycyrrhiza yunnanensis Cheng f. et L. K. Tai.; Glycyrrhiza malensis.

“Yanhusuo,” as used herein, refers to extracts of yanhusuo. It is also known as Rhizoma corydalis yanhusuo and Corydalis yanhusuo W. T. Wang. A member of the botanical group Corydalis is a substance that is capable of providing a similar physiological effect(s) as that provided by yanhusuo in the compositions of the invention, and is preferably selected from a group comprising Rhizoma corydalis yanhusuo; Corydalis yanhusuo W. T. Wang.; Corydalis yanhusuo W. T. Wang (yanhusuo); Corydalis amabilis Migo; Corydalis amabigua Cham. et Schlecht. Var amurensts Maxim; Corydalis turtschaninovii Bess.; Corydalis turtschaninovii f. yanhusuo; Corydalis schangini (Pall.) B.; Corydalis glaucescens Rgl.; Corydalis ledebouriana Kar. Et Kir.

“Baishaoyao,” as used herein, refers to extracts of baishaoyao. It is also known as Radix paeoniae lactiflorae and paeonia lactiflora Pall. A member of the botanical group Paeonia is a substance that is capable of providing a similar physiological effect(s) as that provided by baishaoyao in the compositions of the invention, and is preferably selected from a group comprising Radix paeoniae lactiflorae; Radix paeoniae rubra; Radix paeoniae veitchii; Paeonia lactiflora Pall; Paeonia albriflora Pall; Paeonia veitchii Lynch; Paeonia lactiflora Pall. Var. trichocarpa (Bunge) Stern.; Paeonia obovata. Maxim; Paeonia anomala; Paeonia hybrida Pall; Paeonia veitchii var woodwardii; Paeonia edulis (Salisb.); Paeonia officinalis Thunb.

“Huangqi,” as used herein, refers to extracts of huangqi. It is also known as radix astragali, astragalus membranaceus (Fisch.) Bge and astragalus membranaceus (Fisch.) Bge. mongholicus. A member of the botanical group Astragalus is a substance that is capable of providing a similar physiological effect(s) as that provided by huangqi in the compositions of the invention, and is preferably selected from a group comprising Radix astragali; Radix astragali membranaceus; Astragalus membranaceus (Fisch) Bge; astragalus membranaceus (Fisch.) Bge. mongholicus; Astragalus membranaceus; Astragalus mongholicus Bunge; Astragalus tongolensis Ulbr.; Astragalus chrysopterus Bunge; Astragalus floridus Benth; Astragalus maonwenensis Hsiao; Astragalus yunnanensis Franch; Astragalus ernestii Comb.; Hedysarum polybotrys Hand-Mazz; Hedysarum austrosibiricum B.; Hedysarum limprichtii Ulbr.; Hedysarum viciodes Turez var taipeicum (Hand-Mazz) Lin.; Hedysarum sikkimense Benth.

“Gegen,” as used herein, refers to extracts of gegen. It is also known as radix puerariae and pueraria lobata Ohwi. A member of the botanical group Pueraria is a substance that is capable of providing a similar physiological effect(s) as that provided by gegen in the compositions of the invention, and is preferably selected from a group comprising Radix puerariae; Pueraria lobata (Willd.) Ohwi; Pueraria lobata var. chinensis; Pueraria lobata var. thomsonii; Pueraria lobata; Pueraria lobata ((Willd.)Ohwi.); Pueraria lobata thomsonii ((Benth.)Maesen.); Pueraria montana lobata; Pueraria montana var. chinensis; Pueraria montana var. lobata; Pueraria montana chinensis; Pueraria omeiensis Wang et Tang; Pueraria pseudohirsuta Tang et Wang; Pueraria thomsonii (Benth.); Pueraria thomsonii; Pueraria thunbergiana (Sieb.et Zucc)Benth; Pueraria thunbergiana ((Siebold.&Zucc.)Benth; Pueraria thunbergiana; Pueraria hirsute; Pueraria hirsuta; Pueraria hirsuta ((Thunb.)Matsum.); Pueraria collettii; Pueraria siamica; Pueraria stricta; Pachyrhizus thunbergianus; Dolichos hirsutus; Neustanthus chinensis; Dolichos lobatus; Dolichos lobatus (Willd.).

“Chongweizi,” as used herein, refers to extracts of chongweizi. It is also known as Fructus leonuri and Leonurus japonicus Houtt. A member of the botanical group Leonurus is a substance that is capable of providing a similar physiological effect(s) as that provided by chongweizi in the compositions of the invention, and is preferably selected from a group comprising Fructus leonuri; Leonurus japonicus Houtt.; Leonurus japonicus.

“Zhushizi,” as used herein, refers to extracts of zhushizi. It is also known as Fructus broussonetiae and Broussonetia papyrifera (L.) Vent. A member of the botanical group Broussonetia is a substance that is capable of providing a similar physiological effect(s) as that provided by zhushizi in the compositions of the invention, and is preferably selected from a group comprising Fructus broussonetiae; Broussonetia papyrifera; Broussonetia papyrifera (L.) Vent.

“Rice flour powder,” as used herein, refers to the additive known to persons of skill in the art as such.

“Selenium,” as used herein, refers to the form(s) of the mineral known to persons of skill in the art to be therapeutically effective in the body of the individual. It is preferably provided as selenium aminoate.

“Vitamin B-2,” as used herein, refers to the riboflavin member of the Vitamin B series or complex.

“Vitamin C,” as used herein, refers to ascorbic acid and salts thereof.

“Vitamin E,” as used herein, refers to D alpha-tocopherol, preferably in its succinate form.

“L-carnitine,” as used herein, refers to the biochemical molecule known as such by persons of skill in the art. It is generally known as a zwitterionic compound formed from lysine. It also refers to related molecules such as L-acetyl-carnitine.

“Zexie,” as used herein, refers to extracts of zexie. It is also known as rhizoma alismatis orientalitis and alisma plantagoaquatica L. A member of the botanical group Alisma is a substance that is capable of providing a similar physiological effect(s) as that provided by zexie in the compositions of the invention, and is preferably selected from a group comprising Rhizoma alismatis orientalitis; Alisma plantagoaquatica L; Alisma plantago-aquatica L. var. orientale Sam.; Alisma orientalitis (Sam.) Juzep.; Alisma parviflorum (Pursh.); Alisma subcordatum (Raf.); Alisma triviale (Pursh.); Alisma brevipes; Alisma plantago; Alisma plantago-aquatica ssp. Brevipes; Alisma plantago-aquatica ssp. subcordatum; Alisma plantagoaquatica var. americanum; Alisma plantago-aquatica var. brevipes; Alisma plantago-aquatica var. parviflorum.

“Zinc,” as used herein, refers to the form(s) of the mineral known to persons of skill in the art to be therapeutically effective in the body of the individual. It is preferably provided as zinc gluconate.

“Extract,” as used herein, refers to the substances obtained from the specified source plant, or parts thereof (for e.g., root, bark, leaves). Any method of extraction that yields extracts that retain the biological activity of the substances contained in the extract source can be used to produce extracts used in this invention. Preferably, the ingredients of the compositions of the present invention are extracted as an aqueous solution. The extraction is preferably performed under conditions of high pressure, preferably from 0.5 to 12 bar, more preferably 1 to 10 bar, most preferably 3 to 7 bar, and preferably at elevated temperatures (preferably within a range of 15° C. to 120° C., more preferably 30° C. to 100° C., most preferably 45° C. to 75° C.). The extract is preferably treated to yield a form suitable for mixing of two or more substances. The form is preferably a dried powder. The powder form is yielded from preferably at least about a 1:10, more preferably at least about a 1:8, most preferably at least about a 1:5 concentrate of the starting solution. Concentration to powder form is preferably achieved by evaporation to yield a dried powder form. The extracts used in this invention can also be obtained from commercial sources such as Sun Ten Laboratories (Irvine, Calif.), Qualiherb (Cerritos, Calif.), Mayway (Oakland, Calif.), Ming Tong Herb (Oakland, Calif.) and Acta (Sunnyvale, Calif.). It is understood that any method or conditions known in the art to yield extracts comparable in therapeutic effectiveness to those produced by the preceding preferred extraction method can be used for the purposes of this invention.

Formulation of the Compositions

Each substance contained in the compositions provided by this invention is provided in an amount that lies within specific quantitative ranges herein disclosed to be effective for treating eye discomfort.

According to the present invention, an effective amount of a composition comprises preferably from 12 to 200 mg, more preferably 25 to 150 mg, most preferably 50 to 100 mg of a member of the botanical group poria cocos (such as fuling); preferably from 5 to 100 mg, more preferably 25 to 75 mg, most preferably 50 to 60 mg of a member of the botanical group Cassia (such as jueminzi); preferably 35 to 400 mg, more preferably 75 to 300, most preferably 125 to 225 mg of a member of the botanical group Morus (such as sang ye); 12 to 200 mg, more preferably 25 to 150 mg, most preferably 50 to 100 mg of a member of the botanical group Dioscorea (such as shanyao); 12 to 200 mg, more preferably 25 to 150 mg, most preferably 50 to 100 mg of a member of the botanical group officinale (such as shanzhuyu); and/or preferably 25 to 1200 mg, more preferably 75 to 1000 mg, most preferably 250 to 750 mg of a member of the botanical group Rehmannia (such as shudihaung).

In other embodiments, an effective amount of a composition comprises in some instances from 12 to 200 mg, some instances 25 to 150 mg, some instances 50 to 100 mg of a member of the botanical group poria cocos (such as fuling); in some instances from 12 to 200 mg, some instances 25 to 150 mg, some instances 50 to 100 mg of a member of the botanical group Leonurus (such as chongweizi); in some instances from 12 to 200 mg, some instances 25 to 150 mg, some instances 50 to 100 mg of a member of the botanical group Broussonetia (such as zhushizi); in some instances from 12 to 200 mg, some instances 25 to 150 mg, some instances 50 to 100 mg of a member of the botanical group Salvia (such as danshen); in some instances from 25 to 1200 mg, some instances 75 to 1000 mg, some instances 250 to 750 mg of a member of the botanical group Lycium (such as gouqizhi); in some instances from 25 to 1200 mg, some instances 75 to 1000 mg, some instances 250 to 750 mg of a member of the botanical group Panax (such as shanqi); in some instances from 12 to 200 mg, some instances 25 to 150 mg, some instances 50 to 100 mg of a member of the botanical group Chrysanthemum (such as juhua); in some instances from 12 to 200 mg, some instances 25 to 150 mg, some instances 50 to 100 mg of a member of the botanical group Paeonia suffruticosa (such as mudanpi); in some instances from 12 to 200 mg, some instances 25 to 150 mg, some instances 50 to 100 mg of a member of the botanical group Dioscorea (such as shanyao); in some instances from 12 to 200 mg, some instances 25 to 150 mg, some instances 50 to 100 mg of a member of the botanical group officinale (such as shanzhuyu); in some instances from 25 to 1200 mg, some instances 75 to 1000 mg, some instances 250 to 750 mg of a member of the botanical group Rehmannia (such as shudihaung); in some instances from 12 to 200 mg, some instances 25 to 150 mg, some instances 50 to 100 mg of a member of the botanical group Alisma (such as zexie); in some instances from 25 to 1200 mg, some instances 75 to 1000 mg, some instances 250 to 750 mg of a member of the botanical group Glycyrrhiza (such as gancao); and/or in some instances from 25 to 1200 mg, some instances 75 to 1000 mg, some instances 250 to 750 mg of a member of the botanical group Corydalis (such as yanhusuo).

In other embodiments, an effective amount of a composition comprises in some instances from 12 to 200 mg, some instances 25 to 150 mg, some instances 50 to 100 mg of a member of the botanical group poria cocos (such as fuling); in some instances from 12 to 200 mg, some instances 25 to 150 mg, some instances 50 to 100 mg of a member of the botanical group Pueraria (such as gegen); in some instances from 12 to 200 mg, some instances 25 to 150 mg, some instances 50 to 100 mg of a member of the botanical group Broussonetia (such as zhushizi); in some instances from 12 to 200 mg, some instances 25 to 150 mg, some instances 50 to 100 mg of a member of the botanical group Salvia (such as danshen); in some instances from 12 to 200 mg, some instances 25 to 150 mg, some instances 50 to 100 mg of a member of the botanical group Paeonia (such as baishaoyao); in some instances from 25 to 1200 mg, some instances 75 to 1000 mg, some instances 250 to 750 mg of a member of the botanical group Lycium (such as gouqizhi); in some instances from 25 to 1200 mg, some instances 75 to 1000 mg, some instances 250 to 750 mg of a member of the botanical group Panax (such as shanqi); in some instances from 12 to 200 mg, some instances 25 to 150 mg, some instances 50 to 100 mg of a member of the botanical group Chrysanthemum (such as juhua); in some instances from 12 to 200 mg, some instances 25 to 150 mg, some instances 50 to 100 mg of a member of the botanical group Paeonia suffruticosa (such as mudanpi); in some instances from 12 to 200 mg, some instances 25 to 150 mg, some instances 50 to 100 mg of a member of the botanical group Dioscorea (such as shanyao); in some instances from 12 to 200 mg, some instances 25 to 150 mg, some instances 50 to 100 mg of a member of the botanical group officinale (such as shanzhuyu); in some instances from 25 to 1200 mg, some instances 75 to 1000 mg, some instances 250 to 750 mg of a member of the botanical group Rehmannia (such as shudihaung); in some instances from 12 to 200 mg, some instances 25 to 150 mg, some instances 50 to 100 mg of a member of the botanical group Alisma (such as zexie); in some instances from 25 to 1200 mg, some instances 75 to 1000 mg, some instances 250 to 750 mg of a member of the botanical group Glycyrrhiza (such as gancao); in some instances from 25 to 1200 mg, some instances 75 to 1000 mg, some instances 250 to 750 mg of a member of the botanical group Corydalis (such as yanhusuo); and/or in some instances from 25 to 1200 mg, some instances 75 to 1000 mg, some instances 250 to 750 mg of a member of the botanical group Astragalus (such as huangqi).

In the various embodiments of the invention, the compositions further comprise from 12 to 200 mg, more preferably 25 to 150 mg, most preferably 50 to 100 mg of a member of the botanical group Vaccinium (such as bilberry); 12 to 200 mg, more preferably 25 to 150 mg, most preferably 50 to 100 mg of a member of the botanical group Salvia (such as danshen); preferably 75 to 1200 mg, more preferably 200 to 1000 mg, most preferably 350 to 750 mg of a member of the botanical group Lycium (such as gouqizhi); preferably 50 to 700 mg, more preferably 150 to 500 mg, most preferably 300 to 400 mg of a member of the botanical group Chrysanthemum (such as juhua); 12 to 200 mg, more preferably 25 to 150 mg, most preferably 50 to 100 mg of a member of the botanical group Tribulus (such as baijili); preferably 15 to 250 mg, more preferably 50 to 150 mg, most preferably 75 to 100 mg beta-carotene, preferably 0.5 to 70 mg preferably 5 to 80 mg, more preferably 15 to 60 mg, most preferably 40 to 50 mg copper gluconate or cupric oxide; preferably 5 to 100 mg, more preferably 20 to 75 mg, most preferably 35 to 50 mg of a member of the botanical group Eriocaulon (such as gujingcao); preferably 7 to 120 mg, more preferably 25 to 100 mg, most preferably 50 to 75 mg magnesium stearate NF; 12 to 200 mg, more preferably 25 to 150 mg, most preferably 50 to 100 mg of a member of the botanical group Paeonia suffruticosa (such as mudanpi); preferably 5 to 100 mg, more preferably 25 to 75 mg, most preferably 50 to 60 mg rice flour powder; preferably 1 to 70 mg, preferably 4 to 80 mg, more preferably 12 to 65 mg, most preferably 25 to 50 mg selenium aminoate or sodium selenate; preferably 1 to 60 mg, preferably 10 mg to 600 mg, more preferably 50 mg to 500 mg, most preferably 200 mg to 400 mg L-carnitine; preferably 3 to 75 mg, more preferably 12 to 50 mg, most preferably 25 to 40 mg vitamin B-2; preferably 10 to 700 mg, preferably 40 to 800 mg, more preferably 120 to 600 mg, most preferably 250 to 500 mg vitamin C; preferably 10 to 700 mg, preferably 50 to 850 mg, more preferably 150 to 650 mg, most preferably 300 to 500 mg vitamin E; 12 to 200 mg, more preferably 25 to 150 mg, most preferably 50 to 100 mg of a member of the botanical group Alisma (such as zexie); and/or preferably 10 to 650 mg, preferably 40 to 60.0 mg, more preferably 120 to 500 mg, most preferably 250 to 400 mg zinc gluconate or zinc oxide.

For example, a composition may comprise preferably 25 to 1200 mg, more preferably 75 to 1000 mg, most preferably 250 to 750 mg of a member of the botanical group Rehmannia (such as shudihaung) and preferably 75 to 1200 mg, more preferably 200 to 1000 mg, most preferably 350 to 750 mg of a member of the botanical group Lycium (gouqizhi). In another example, a composition may comprise preferably 50 to 700 mg, more preferably 150 to 500 mg, most preferably 300 to 400 mg of a member of the botanical group Chrysanthemum (such as juhua) and preferably 35 to 400 mg, more preferably 75 to 300, most preferably 125 to 225 mg of a member of the botanical group Morus (such as sang ye).

Selection of suitable members of a particular botanical group to be included in a composition can be achieved using methods known in the art. For example, a suitable member of the botanical group Cassia would be expected to be capable of providing a similar activity and/or function as that provided by jueminzi in a composition of the invention. Such a member can be selected based on, for example, whether it is known, shown and/or suspected to possess said similar activity and/or function. Thus, for example, a determination of whether a candidate substance can be a member of the botanical group Cassia can be done based on, for example, a similar pharmacological or medicinal classification for both the candidate substance and jueminzi. However, the activity and/or function provided by a particular substance, such as jueminzi, need not be identified or specified. A determination of whether a candidate substance can be a member of a particular botanical group, for example the Cassia group, can also be empirical, for example, by substituting said candidate substance for jueminzi in a composition, and assessing the relevant therapeutic effect(s) of the composition. Such a determination can be done using methods and techniques known in the art.

According to this invention, the compositions can be formulated in whatever physical form that retains the efficacy of the composition for treating eye discomfort or disease. Preferably, the compositions are packaged in the form of capsules. The capsules are preferably of size “0”, “00”, “000”, “1”, “2”, “3” or “4.” A preferred method for packaging into capsules involves mixing substances (extracts, vitamin and minerals) that are preferably in powder form. The substances are preferably mixed to at least 30%, more preferably to at least 60%, even more preferably to at least 90% mixture consistency, and most preferably to homogeneity. The substances in powder form are provided in the initial mixture at ratios according to the effective quantities disclosed above. Methods for mixing the substances are known in the art, including, but not limited to, stirring, agitation or vibration achieved manually or through the aid of a machine. A preferred mixing machine is a V-mixer, preferably of 100 to 1400-liter size, more preferably of 150 to 1300-liter size, and most preferably of 200 to 1200-liter size. Preferably, the resulting powder mixture is filtered to screen out particulates (i.e., anything that a person of skill in the art would recognize to be larger than powder size). A preferred filter is a 1/20-inch particle size filter. Preferably, the filtered mixture is packaged into capsules according to the weight desired for each capsule. Preferably, the capsule is of size “00”. The weight of mixture per capsule is preferably from 5 mg to 1000 mg, more preferably 100 mg to 800 mg, more preferably about 450 mg to about 750 mg, even more preferably 400 mg to 700 mg. It is understood that other physical forms of the compositions of this invention suitable for administration to an individual can also be used, including, for example, tablets, salves or liquids, as long as the compositions can be delivered to the target tissues in the body where the compositions in the preferred form described above exert their effects.

The ingredients of the compositions can be mixed with pharmaceutically acceptable solvents, excipients and/or filler substances. These materials are known in the art, and are described in sources such as Remington 's Pharmaceutical Sciences, 18th Edition, Mack Publishing (1990).

Administration of Compositions

Compositions in any of the physical forms described above can be administered by any method known to one of skill in the art, but oral administration is preferred. The compositions are preferably administered in capsule form.

An effective amount of a composition is provided preferably in from 1 to 8 administrations, more preferably in from 2 to 6 administrations, and most preferably in from 3 to 5 administrations. In some embodiments, an effective amount of a composition may be provided, for example, from about any of the following: 1 to 40, 2 to 30, 2 to 20, 3 to 15, 4 to 12, 5 to 30, 10-20 administrations. Administration of an effective amount is preferably completed within 24 hours. A composition can be ingested alone, or with any other substance, such as a liquid, that aids ingestion of the compositions. Ingestion of the compositions can be before or after food consumption.

### EXAMPLES

### Example 1

An Illustrative Example of the Formulation of A Single “00” Capsule, and the Production Thereof

A composition shown to be effective for treating eye discomfort contained substances in the indicated quantities as listed in Table 1.

TABLE 1

| SUBSTANCE [Commercial source] | AMOUNT (mg) |
|---|---|
| Fuling [Qualiherb, Cerritos, CA] | 16.7 |
| Jueminzi [Qualiherb, Cerritos, CA] | 8.3 |
| Sang ye [Qualiherb, Cerritos, CA] | 33.3 |
| Shanyao [Qualiherb, Cerritos, CA] | 16.7 |
| Shanzhuyu [Mayway, Oakland, CA] | 16.7 |
| Shudihaung [Qualiherb, Cerritos, CA] | 100.0 |
| Bilberry [Acta, Sunnyvale, CA] | 16.7 |
| Danshen [Qualiherb, Cerritos, CA] | 16.7 |
| Gouqizhi [Qualiherb, Cerritos, CA] | 100.0 |
| Juhua [Qualiherb, Cerritos, CA] | 66.7 |
| Baijili [Qualiherb, Cerritos, CA] | 16.7 |
| Beta-carotene [Acta, Sunnyvale, CA] | 22.5 |
| Copper gluconate [Acta, Sunnyvale, CA] | 7.1 |
| Gujincao [Qualiherb, Cerritos, CA] | 8.3 |
| Magnesium stearate NF [Acta, | 10.0 |
| Sunnyvale, CA] | |
| Mudanpi [Qualiherb, Cerritos, CA] | 16.7 |
| Rice flour powder [Acta, Sunnyvale, CA] | 8.3 |
| Seleniuum aminoate [Acta, Sunnyvale, | 6.7 |
| CA] | |
| Vitamin B-2 (riboflavin) [Acta, | 5.0 |
| Sunnyvale, CA] | |
| Vitamin C (ascorbic acid) [Acta, | 66.7 |
| Sunnyvale, CA] | |
| Vitamin E (D Alpha-tocopherol | 76.0 |
| succinate) [Acta, Sunnyvale, CA] | |
| Zexie [Acta, Sunnyvale, CA] | 16.7 |
| Zinc gluconate [Acta, Sunnyvale, CA] | 57.7 |

Capsules containing the composition above were manufactured according to the method used by the commercial manufacturer, Acta (Sunnyvale, Calif.). Briefly, the substances listed above, in powder form and obtained from the commercial sources indicated, were mixed in input amounts in accordance to the ratio of the substances in the composition as a whole. Mixing was accomplished with a V-mixer, grinding for 15 to 30 minutes, at a speed of 15 to 30 rpm (rounds per minute), to produce a homogenous mixture of the input substances. Particulates (non-powder forms) were then filtered out with a 1/20-inch particle size filter that separated particulates from the powder. 694.97 mg of the filtered mixture was then packaged into each size “00” capsule.

### Example 2

### Illustrative Example of Treatment of Dry Eye Condition

10 individuals with dry eye symptom were treated with the composition exemplified in Example 1. They were instructed to fill out a clinical questionnaire to assess efficacy of treatment with the composition over time. The individuals were administered the composition for the duration and in the amount (capsules/day) as indicated in Table 2.

TABLE 2
Dosage Amount

| | Day 3-5 | Day 5-7 | Day 8-14 | Day 15-21 | Day 22-28 | Day 29-35 |
|---|---|---|---|---|---|---|
| Individual | (capsules/ | (capsules/ | (capsules/ | (capsules/ | (capsules/ | (capsules/ |
| No. | day) | day) | day) | day) | day) | day) |
| 1 | 2 | 2 | 2 | 2 | 2 | |
| 2 | 6 | 6 | 2 | 1 | 1 | 1 |
| 3 | 1 | 1 | 1 | 1 | 1 | 1 |
| 4 | 2 | 2 | 2 | 2 | 2 | 2 |
| 5 | 1 | 1 | 1 | 1 | 1 | 1 |
| 6 | 6 | 6 | 6 | 6 | 6 | 6 |
| 7 | 4 | 4 | 4 | 4 | 4 | |
| 8 | 4 | 4 | 4 | 4 | 4 | |
| 9 | 4 | 4 | 4 | 4 | 4 | |
| 10 | 4 | 4 | 4 | 4 | 4 | |

The results of the study are summarized in Table 3.

Severity of dry eye symptom was graded on a scale of 0 to 3.0 was defined as normal eye condition, 1 was occasional symptoms, 2 was frequent symptoms and 3 was constant dry eye symptoms. Individuals were instructed to assess amount of tear production by their eyes as a primary indication of dry eye condition.

TABLE 3
Severity of Dry Eye Condition

| Indi- | | | | | | | Day | Day |
|---|---|---|---|---|---|---|---|---|
| vidual | Before | Day | Day | Day | Day | Day | 29- | 36- |
| No. | use | 3-5 | 5-7 | 8-14 | 15-21 | 22-28 | 35 | 42 |
| 1 | 2 | 2 | 1 | 1 | 1 | 1 | 0 | 0 |
| 2 | 3 | 2 | 2 | 1 | 1 | 1 | 1 | 0 |
| 3 | 3 | 2 | 2 | 1 | 1 | 1 | 1 | 0 |
| 4 | 3 | 3 | 3 | 2 | 1 | 1 | 1 | 0 |
| 5 | 3 | 2 | 2 | 1 | 1 | 1 | 1 | 1 |
| 6 | 3 | 2 | 2 | 1 | 1 | 1 | 1 | 0 |
| 7 | 3 | 3 | 2 | 1 | 1 | 1 | | |
| 8 | 2 | 2 | 1 | 1 | 1 | | | |
| 9 | 3 | 3 | 2 | 1 | | | | |
| 10 | 3 | 3 | 2 | 1 | 1 | 1 | | |

The results show a clear and significant decrease, and in some cases complete clearance, of dry eye symptoms following the administration of the composition of Example 1.

### Example 3

### Illustrative Example of Treatment of Eye Strain/Fatigue

Eight of the individuals of Example 2 were also assessed for eye strain/fatigue condition. Results are summarized in Table 4.

Severity of eye strain/fatigue was graded on a scale of 0 to 3.0 was defined as normal eye condition, 1 was occasional symptoms, 2 was frequent symptoms and 3 was constant eye strain/fatigue. Individuals were instructed to assess “burning” feeling and “sandy grittiness” of the eyes as primary indications of eye strain/fatigue.

TABLE 4
Severity of Eye Strain/Fatigue

| Indi- | | | | | | | Day | Day |
|---|---|---|---|---|---|---|---|---|
| vidual | Before | Day | Day | Day | Day | Day | 29- | 36- |
| No. | use | 3-5 | 5-7 | 8-14 | 15-21 | 22-28 | 35 | 42 |
| 1 | 2 | 2 | 1 | 1 | 1 | 1 | 0 | 0 |
| 2 | 3 | 2 | 2 | 1 | 1 | 1 | 1 | 0 |
| 3 | 3 | 2 | 2 | 1 | 1 | 1 | 1 | 0 |
| 4 | | | | | | | | |
| 5 | | | | | | | | |
| 6 | 3 | 2 | 2 | 1 | 1 | 1 | 1 | 0 |
| 7 | 3 | 3 | 2 | 1 | 1 | 1 | | |
| 8 | 2 | 2 | 1 | 1 | 1 | | | |
| 9 | 3 | 3 | 2 | 1 | | | | |
| 10 | 3 | 3 | 2 | 1 | 1 | 1 | | |

The results show a clear and significant improvement, and in some cases complete clearance, of eye strain/fatigue in individuals administered the composition described in Example 1.

### Example 4

### Illustrative Example of Treatment of Eye Soreness

Seven of the individuals of Example 2 were also assessed for eye soreness. Results are summarized in Table 5.

Severity of eye soreness was graded on a scale of 0 to 3.0 was defined as normal eye condition, 1 was occasional symptoms, 2 was frequent symptoms and 3 was constant eye soreness. Individuals were instructed to assess “pain-like” sensations of the eyes as primary indications of eye soreness.

TABLE 5
Severity of Eye Soreness

| Indi- | | | | | | | Day | Day |
|---|---|---|---|---|---|---|---|---|
| vidual | Before | Day | Day | Day | Day | Day | 29- | 36- |
| No. | use | 3-5 | 5-7 | 8-14 | 15-21 | 22-28 | 35 | 42 |
| 1 | 2 | 2 | 1 | 1 | 1 | 1 | 0 | 0 |
| 2 | 3 | 2 | 2 | 1 | 1 | 1 | 1 | 0 |
| 3 | 3 | 2 | 2 | 1 | 1 | 1 | 1 | 0 |
| 4 | | | | | | | | |
| 5 | | | | | | | | |
| 6 | 3 | 2 | 2 | 1 | 1 | 1 | 1 | 0 |
| 7 | | | | | | | | |
| 8 | 2 | 2 | 1 | 1 | 1 | | | |
| 9 | 3 | 3 | 2 | 1 | | | | |
| 10 | 3 | 3 | 2 | 1 | 1 | 1 | | |

The results show a clear and significant improvement, and in some cases complete clearance, of eye soreness in individuals administered the composition described in Example 1.

### Example 5

### Illustrative Example of Treatment of Watery Eye Condition

Three of the individuals of Example 2 were also assessed for watery eye condition. Results are summarized in Table 6.

Severity of watery eye condition was graded on a scale of 0 to 3.0 was defined as normal eye condition, 1 was occasional symptoms, 2 was frequent symptoms and 3 was constant water eye condition. Individuals were instructed to assess amount of tear production by the eyes as a primary indication of watery eye condition.

TABLE 6
Severity of Watery Eye Condition

| Indi- | | | | | | | Day | Day |
|---|---|---|---|---|---|---|---|---|
| vidual | Before | Day | Day | Day | Day | Day | 29- | 36- |
| No. | use | 3-5 | 5-7 | 8-14 | 15-21 | 22-28 | 35 | 42 |
| 1 | | | | | | | | |
| 2 | | | | | | | | |
| 3 | | | | | | | | |
| 4 | | | | | | | | |
| 5 | | | | | | | | |
| 6 | | | | | | | | |
| 7 | | | | | | | | |
| 8 | 2 | 2 | 1 | 1 | 1 | | | |
| 9 | 2 | 2 | 1 | 1 | | | | |
| 10 | 2 | 2 | 2 | 1 | 1 | 1 | | |

The results show a clear and significant improvement of watery eye condition in individuals administered the composition described in Example 1.

### Example 6

### Illustrative Example of Treatment of Contact Lens Discomfort

One of the individuals of Example 2 was also assessed for contact lens discomfort. Results are summarized in Table 7.

Severity of contact lens discomfort was graded on a scale of 0 to 3.0 was defined as normal eye condition, 1 was occasional symptoms, 2 was frequent symptoms and 3 was constant contact lens discomfort. The individual was instructed to assess conditions of dry eye, eye strain/fatigue, eye soreness and/or watery eye (all conditions as defined in the preceding Examples) as primary indications of discomfort associated with the wearing of contact lens.

TABLE 7
Severity of Contact Lens Discomfort

| Indi- | | | | | | | Day | Day |
|---|---|---|---|---|---|---|---|---|
| vidual | Before | Day | Day | Day | Day | Day | 29- | 36- |
| No. | use | 3-5 | 5-7 | 8-14 | 15-21 | 22-28 | 35 | 42 |
| 1 | | | | | | | | |
| 2 | | | | | | | | |
| 3 | | | | | | | | |
| 4 | 3 | 3 | 3 | 2 | 1 | 1 | | |
| 5 | | | | | | | | |
| 6 | | | | | | | | |
| 7 | | | | | | | | |
| 8 | | | | | | | | |
| 9 | | | | | | | | |
| 10 | | | | | | | | |

The results indicate that the composition is also effective in treating eye discomfort associated with the wearing of contact lenses.

### Example 7

### Illustrative Example of Treatment of Eye Discomfort

A randomized, double-blinded and controlled study is performed. A control group is included that uses placebo capsules. Dosage amount and schedule are 1 to 6 capsules (of the composition of Example 1) daily. At least 20 individuals are tested, randomly assigned in approximately equal number to treatment (administered the composition of Example 1) and control groups. They are evaluated to suffer from eye discomfort symptoms, such as dry eye, eye strain/fatigue, eye soreness, and/or watery eye.

The study is carried out for at least 1 week. During the treatment course, a dosage amount selected from the range of 1 to 6 capsules is administered to each individual once or multiple times daily, not exceeding 6 capsules per day. Capsules are administered before or after food consumption.

A clinical questionnaire is used to evaluate individuals' eye discomfort symptoms. A clinical coordinator and/or physician evaluates the individuals' eye discomfort symptoms and fills out the questionnaire. The criteria for assessing severity of symptoms include those described in the preceding Examples. Evaluations can be performed daily, or more or less frequently depending on statistical or clinical (ability to detect or track symptomatic improvements) need.

Assessment of symptoms is divided into 4 grades: (1) clinical cure, as defined as free of symptoms; (2) significant efficacy, as defined as significantly improved symptoms (e.g., reduction of more than 3 points on any one of discomfort scoring scales); (3) efficacy, as defined as partially improved (e.g. reduction of more than 1 point on any one of discomfort scoring scales); and (4) non-efficacy, as defined as no improvement in symptoms.

### Example 8

### A Second Illustrative Example of Treatment of Eye Discomfort

A multi-center, double-blind, randomized, parallel, placebo-controlled study was conducted. The study was of four weeks in duration. Prior to treatment, subjects were asked to note their average symptom scores, which were recorded to establish an accurate baseline. Two and four weeks following treatment, subjects were asked to record their symptom scores. In this study, the composition of Example 1 was co-administered with an enhancing composition (which by itself is not efficacious in treating eye discomfort conditions, which is disclosed in copending application entitled “Compositions And Methods For Enhancing Therapeutic Effects” (U.S. Provisional Application Ser. No. 60/208,990; U.S. patent application Ser. No. 09/872,410, now U.S. Pat. No. 6,521,269, priority application filed even date with priority application of present application), which is hereby incorporated in its entirety by reference. Each capsule containing the enhancing composition contained substances described in Table 8 below.

TABLE 8

| SUBSTANCE [Commercial Source] | AMOUNT (mg) |
|---|---|
| Xuejie [Min Tong Herb, Oakland, CA] | 133 |
| Yanhusuo [Mayway, Oakland, CA] | 83 |
| Baishaoyao [Qualiherb, Cerritos, CA] | 83 |
| Shanqi [Sun Ten, Irvine, CA] | 250 |
| Gancao [Qualiherb, Cerritos, CA] | 83 |
| White willow bark [Acta, Sunnyvale, CA] | 10 |
| Black cohosh root [Acta, Sunnyvale, CA] | 10 |
| L-carnitine [Acta, Sunnyvale, CA] | 50 |
| Vitamin E (D Alpha-Tocopherol succinate) | 21.19 |
| [Acta, Sunnyvale, CA] | |
| Vitamin C [Acta, Sunnyvale, CA] | 30 |

Subjects were placed on active treatment or placebo for four weeks. During the treatment phase, each subject was administered 2 capsules of the composition of Example 1 and 2 capsules of the enhancing composition of Table 8 two times daily between or after meals for two weeks, followed by 2 capsules of the composition of Example 1 (without the enhancing composition) two times daily between or after meals for another two weeks. The control subjects were administered placebo capsules according to the same treatment and dosage regimen.

Inclusion Criteria

Study subjects were selected based on a number of set criteria, including:

(1) a natural or corrected visual acuity of 20/25 or better;

(2) Zone Quick test of greater than 5 mm in 15 seconds;

(3) Tear Breakup test of greater than 5 seconds;

(4) have had one of the eye conditions listed below for at least a month, of severity greater than 3 on a subjective 1-10 scale of eye discomfort: Conditions:

itchy eyes

red eyes

burning eyes

pain

tearing (watery) eyes

dry eyes

sandy (gritty) eyes

tired/sore/eyestrain or eye fatigue

contact lens discomfort

overall eye discomfort

Assessment of Treatment Effect

(1) Assessment of improvement of abnormal eye symptoms by Tear Breakup Test: Subjects who had abnormal eyes prior to treatment as indicated by a tear breakup test of less than or equal to 10 seconds were assessed by the Tear Breakup Test at two and 4 weeks after treatment. The Tear Breakup Test measures the time between a complete blink and the appearance of the first randomly distributed corneal dry spot. A moistened fluorescein strip is touched lightly against the inferior tarsal conjunctiva. After several blinks, the patient stares ahead without blinking. A cobalt-blue-filtered light is used to illuminate the front surface of the cornea. The time from the blink to the appearance of the black spot (defect) is measured in seconds.

The percentage of subjects showing improvement observed over baseline (prior to treatment) was determined. Results are described in Table 9.

TABLE 9
Percentage of abnormal eyes that improved

| | 2 weeks | 4 weeks |
|---|---|---|
| Treated | 77% | 84% |
| Placebo | 58% | 61% |

(2) Assessment of improvement of abnormal eye symptoms by Zone quick test: Subjects who had abnormal eyes prior to treatment as indicated by a Zone quick test showing equal to or less than 20 mm in dye migration were assessed by the Zone quick test at two and 4 weeks after treatment. The Zone Quick test is a minimally invasive test that measures tear volume by use of a special long strand cotton yellow thread that turns red when exposed to tear. The subject is instructed to look straight ahead. The thread is placed one-third of the distance in from the outer canthus of the lower eyelid. The lower lid is gently pulled down and a folded end of thread is placed on the palpebral conjunctiva. The lid is then released. The subject is instructed to continue primary gaze and to blink normally. The thread is removed 15 seconds later, by gently pulling the lower lid down and removing the thread with an upward motion. The red zone of the thread is measured in millimeters, with a measurement of 10 mm or less indicating abnormally low tear volume and a measurement of 20 mm or greater considered normal.

The percentage of subjects showing improvement observed over baseline (prior to treatment) was determined. Results are described in Table 10.

TABLE 10
Percentage of abnormal eyes that improved

| | 2 weeks | 4 weeks |
|---|---|---|
| Treated | 77% | 83% |
| Placebo | 38% | 53% |

(3) Assessment of improvement of abnormal eye symptom by corneal staining: Subjects who had abnormal eyes as indicated by corneal staining prior to treatment were assessed by corneal staining at two and 4 weeks after treatment. Corneal staining involves microscopic examination of the eye to evaluate integrity of the corneal epithelium. It involves injection of a fluorescence dye into the eye to obtain an ogmective assessment of inflammation of the conjunctiva.

The percentage of subjects showing improvement observed over baseline (prior to treatment) was determined. Results are described in Table 11.

TABLE 11
Percentage of abnormal eyes that improved

| | 2 weeks | 4 weeks |
|---|---|---|
| Treated | 81% | 89% |
| Placebo | 67% | 53% |

The percentages of abnormal eyes that did not improve as assessed by the tests described above were also recorded, and are described in Tables 12, 13 and 14.

TABLE 12
Percentage of abnormal eyes that did not improve
by Tear Breakup Test

| | 2 weeks | 4 weeks |
|---|---|---|
| Treated | 13% | 14% |
| Placebo | 29% | 35% |

TABLE 13
Percentage of abnormal eyes that did not improve
by Zone quick test

| | 2 weeks | 4 weeks |
|---|---|---|
| Treated | 0% | 0% |
| Placebo | 31% | 7% |

TABLE 14
Percentage of abnormal eyes that did not improve
by corneal staining

| | 2 weeks | 4 weeks |
|---|---|---|
| Treated | 19% | 11% |
| Placebo | 0% | 0% |

The percentages of abnormal eyes with a negative response as assessed by the tests described above were also recorded, and are described in Tables 15, 16 and 17.

TABLE 15
Percentage of abnormal eyes with a negative response
by Tear Breakup Test

| | |
|---|---|
| 2 weeks | 4 weeks |

| | | |
|---|---|---|
| Treated | 8% | 2% |
| Placebo | 13% | 6% |

TABLE 16
Percentage of abnormal eyes with a negative response
by Zone quick test

| | 2 weeks | 4 weeks |
|---|---|---|
| Treated | 23% | 10% |
| Placebo | 6% | 12% |

TABLE 17
Percentage of abnormal eyes with a negative response
by corneal staining

| | 2 weeks | 4 weeks |
|---|---|---|
| Treated | 0% | 0% |
| Placebo | 30% | 46% |

The percentages of normal eyes with a negative response as assessed by the tests described above were also recorded, and are described in Tables 18, 19 and 20.

TABLE 18
Percentage of normal eyes with a negative response
by Tear Breakup Test

| | 2 weeks | 4 weeks |
|---|---|---|
| Treated | 2% | 0% |
| Placebo | 0% | 0% |

TABLE 19
Percentage of normal eyes with a negative response
by Zone quick test

| | 2 weeks | 4 weeks |
|---|---|---|
| Treated | 0% | 7% |
| Placebo | 25% | 27% |

TABLE 20
Percentage of normal eyes with a negative response
by corneal staining

| | 2 weeks | 4 weeks |
|---|---|---|
| Treated | 0% | 0% |
| Placebo | 0% | 0% |

As illustrated in the data presented above, the composition of Example 1 is efficacious in treating various eye discomfort conditions, generally without causing significant adverse side effects.

### Example 9

### An Illustrative Example of the Formulation of a Single “00” Capsule, and the Production Thereof

A composition for treating symptoms of age-related macular degeneration contains substances in the indicated quantities as listed in Table 21.

TABLE 21

| SUBSTANCE | AMOUNT (mg) |
|---|---|
| Danshen | 27.8 |
| Chongweizi | 27.8 |
| Fuling | 27.8 |
| Zhushizi | 27.8 |
| Gouqizhi | 55.6 |
| Shanqi | 83.3 |
| Juhua | 27.8 |
| Mudanpi | 27.8 |
| Shanyao | 27.8 |
| Shanzhuyu | 27.8 |
| Shudihaung | 55.6 |
| Zexie | 27.8 |
| Gancao | 55.6 |
| Yanhusuo | 55.6 |
| Beta-carotene | 48 |
| Vitamin E (d-alpha-tocopherol acetate) | 18.3 |
| 1.185 iu/mg | |
| Vitamin C (ascorbic acid) | 25.1 |
| Zinc oxide | 29.5 |
| Cupric oxide | 0.64 |
| Vitamin B-2 (riboflavin) | 1.7 |
| Sodium selenate | 2.2 |
| L - carnitine | 17 |
| Rice powder | 8 |
| Magnesium stearate NF | 8 |

Capsules containing the composition above are manufactured according to the method used by the commercial manufacturer, Acta (Sunnyvale, Calif.). Briefly, the substances listed above, in powder form and obtained from commercial sources, are mixed in input amounts in accordance to the ratio of the substances in the composition as a whole. Mixing was accomplished with a V-mixer, grinding for 15 to 30 minutes, at a speed of 15 to 30 rpm (rounds per minute), to produce a homogenous mixture of the input substances. Particulates (non-powder forms) were then filtered out with a 1/20-inch particle size filter that separated particulates from the powder. 713.4 mg of the filtered mixture was then packaged into each size “00” capsule.

### Example 10

### An Illustrative Example of Treatment of Macular Degeneration

A randomized, double-blinded and controlled study is performed with individuals with early to moderate stages of macular degeneration. A control group is included that uses placebo capsules. At least 10 individuals are tested, randomly assigned in approximately equal number to treatment (administered the composition of Example 9) and control groups.

The study is carried out for at least six months. During the treatment course, patricipants will take 18 capsules (of the composition of Example 9) per day (6 capsules morning, noon and night). Evaluations are scheduled prior to treatment and then after 1 month, 3 months and 6 months of treatment. Their visual acuity is evaluated, cataract and/or glaucoma assessment is made and they are subjected to procedures that include retina eye photos, slit lamp eye exams, amsler grids and evaluation of visual acuity by chart.

### Example 11

### An Illustrative Example of the Formulation of a Single “00” Capsule, and the Production Thereof

A composition for treating symptoms of diabetic retinopathy contains substances in the indicated quantities as listed in Table 22.

TABLE 22

| SUBSTANCE | AMOUNT (mg) |
|---|---|
| Danshen | 27.8 |
| Gegen | 27.8 |
| Fuling | 27.8 |
| Baishaoyao | 27.8 |
| Gouqizhi | 55.6 |
| Shanqi | 83.3 |
| Juhua | 27.8 |
| Yanhusuo | 55.6 |
| Mudanpi | 27.8 |
| Shanyao | 27.8 |
| Shanzhuyu | 27.8 |
| Shudihaung | 55.6 |
| Zexie | 27.8 |
| Gancao | 55.6 |
| Huangqi | 27.8 |
| Beta-carotene | 48 |
| Vitamin E (d-alpha-tocopherol acetate) | 18.3 |
| 1.185 iu/mg | |
| Vitamin C (ascorbic acid) | 25.1 |
| Zinc oxide | 29.5 |
| Cupric oxide | 0.64 |
| Vitamin B-2 (riboflavin) | 1.7 |
| Sodium selenate | 2.2 |
| L - carnitine | 17 |
| Rice powder | 8 |
| Magnesium stearate NF | 8 |

Capsules containing the composition above are manufactured according to the method used by the commercial manufacturer, Acta (Sunnyvale, Calif.). Briefly, the substances listed above, in powder form and obtained from commercial sources, are mixed in input amounts in accordance to the ratio of the substances in the composition as a whole. Mixing was accomplished with a V-mixer, grinding for 15 to 30 minutes, at a speed of 15 to 30 rpm (rounds per minute), to produce a homogenous mixture of the input substances. Particulates (non-powder forms) were then filtered out with a 1/20-inch particle size filter that separated particulates from the powder. 713.4 mg of the filtered mixture was then packaged into each size “00” capsule.

### Example 12

### An Illustrative Example of Treatment of Diabetic Retinopathy

A randomized, double-blinded and controlled study is performed with individuals with early diabetic retinopathy, who are on diabetes medication and whose diabetes is under control. A control group is included that uses placebo capsules. At least 10 individuals are tested, randomly assigned in approximately equal number to treatment (administered the composition of Example 11) and control groups.

The study is carried out for at least six months. During the treatment course, participants will take 18 capsules (of the composition of Example 11) per day (6 capsules morning, noon and night). Evaluations are scheduled prior to treatment and then after 1 month, 3 months and 6 months of treatment. Vital signs of the individual are evaluated by determining blood pressure, pulse, temperature and having the individual fill out a quality of life questionaire. Physiological, biological and neurological parameters are measured. The individual is evaluated using the following procedures: retina eye photos, ankle/brachial index determination, peripheral and autonomic neurometers, EKG and an examination from an ophthalmologist. The individual is also evaluated by tracking all blood and urine chemistry including, for example, a metabolic panel, a lipid panel, average blood glucose, creatine levels, urine protein analysis, complete urinalysis, Hemoglobin A1C, SGPT, CBC, platelet aggregation, microalbumin levels, microalbumin/creatinine ratio, homocysteine level and CRP. Prior to treatment, the individuals are evaluated to establish an accurate baseline.

Although the foregoing invention has been described in some detail by way of illustration and example for purposes of clarity of understanding, it will be apparent to those skilled in the art that certain changes and modifications can be practiced. Therefore, the description and examples should not be construed as limiting the scope of the invention, which is delineated by the appended claims.

We claim:

1. A composition for treating eye discomfort comprising 3 substances selected from the group consisting of a member of the botanical group poria cocos, a member of the botanical group Cassia, a member of the botanical group Morus, a member of the botanical group Dioscorea, a member of the botanical group officinale and a member of the botanical group Rehmannia.

2. The composition of claim 1, further comprising at least one substance selected from the group consisting of a member of the botanical group Vaccinium, a member of the botanical group Salvia, a member of the botanical group Lycium, a member of the botanical group Chrysanthemum, a member of the botanical group Tribulus, beta-carotene, copper, a member of the botanical group Eriocaulon, magnesium, a member of the botanical group Paeonia suffruticosa, rice flour powder, selenium, vitamin B-2, vitamin C, vitamin E, a member of the botanical group Alisma and zinc.

3. The composition of claim 1, further comprising a member of the botanical group Vaccinium, a member of the botanical group Salvia, a member of the botanical group Lycium, a member of the botanical group Chrysanthemum, a member of the botanical group Tribulus, beta-carotene, copper, a member of the botanical group Eriocaulon, magnesium, a member of the botanical group Paeonia suffruticosa, rice flour powder, selenium, vitamin B-2, vitamin C, vitamin E, a member of the botanical group Alisma and zinc.

4. The composition of claim 1, wherein the member of the botanical group poria cocos is fuling, the member of the botanical group Cassia is jueminzi, the member of the botanical group Morus is sang ye, the member of the botanical group Dioscorea is shanyao, the member of the botanical group officinale is shanzhuyu, and the member of the botanical group Rehmannia is shudihaung.

5. The composition of claim 2, wherein the member of the botanical group poria cocos is fuling, the member of the botanical group Cassia is jueminzi, the member of the botanical group Morus is sang ye, the member of the botanical group Dioscorea is shanyao, the member of the botanical group officinale is shanzhuyu, the member of the botanical group Rehmannia is shudihaung, the member of the botanical group Vaccinium is bilberry, the member of the botanical group Salvia is danshen, the member of the botanical group Lycium is gouqizhi, the member of the botanical group Chrysanthemum is juhua, the member of the botanical group Tribulus is baijili, the member of the botanical group Eriocaulon is gujincao, the member of the botanical group Paeonia suffruticosa is mudanpi, and the member of the botanical group Alisma is zexie.

6. A composition for treating eye discomfort comprising a member of the botanical group poria cocos, a member of the botanical group Cassia, a member of the botanical group Morus, a member of the botanical group Dioscorea, a member of the botanical group officinale and a member of the botanical group Rehmannia.

7. The composition of claim 6, further comprising at least one substance selected from the group consisting of a member of the botanical group Vaccinium, a member of the botanical group Salvia, a member of the botanical group Lycium, a member of the botanical group Chrysanthemum, a member of the botanical group Tribulus, beta-carotene, copper, a member of the botanical group Eriocaulon, magnesium, a member of the botanical group Paeonia suffruticosa, rice flour powder, selenium, vitamin B-2, vitamin C, vitamin E, a member of the botanical group Alisma and zinc.

8. The composition of claim 6, further comprising a member of the botanical group Vaccinium, a member of the botanical group Salvia, a member of the botanical group Lycium, a member of the botanical group Chrysanthemum, a member of the botanical group Tribulus, beta-carotene, copper, a member of the botanical group Eriocaulon, magnesium, a member of the botanical group Paeonia suffruticosa, rice flour powder, selenium, vitamin B-2, vitamin C, vitamin E, a member of the botanical group Alisma and zinc.

9. A composition for treating eye disease comprising a member of the botanical group Poria cocos, a member of the botanical group Salvia, a member of the botanical group Leonurus, a member of the botanical group Broussonetia, a member of the botanical group Lycium, a member of the botanical group Panax, a member of the botanical group Chrysanthemum, a member of the botanical group Paeonia suffruticosa, a member of the botanical group Dioscorea, a member of the botanical group officinale, a member of the botanical group Rehmannia, a member of the botanical group Alisma, a member of the botanical group Glycyrrhiza and a member of the botanical group Corydalis.

10. The composition of claim 9 further comprising at least one substance selected from the group consisting of beta-carotene, copper, magnesium, rice flour powder, selenium, vitamin B-2, vitamin C, vitamin E, L-carnitine and zinc.

11. The composition of claim 10 comprising danshen, chongweizi, fuling, zhushizi, gouqizhi, shanqi, juhua, mudanpi, shanyao, shanzhuyu, shudihaung, zexie, gancao, yanhusuo, beta-carotene, copper, magnesium, rice flour powder, selenium, vitamin B-2, vitamin C, vitamin E, L-carnitine and zinc.

12. A composition for treating eye disease comprising a member of the botanical group Poria cocos, a member of the botanical group Salvia, a member of the botanical group Puerariae, a member of the botanical group Astragalus, a member of the botanical group Lycium, a member of the botanical group Panax, a member of the botanical group Chrysanthemum, a member of the botanical group Paeonia suffruticosa, a member of the botanical group Dioscorea, a member of the botanical group Paeonia, a member of the botanical group officinale, a member of the botanical group Rehmannia, a member of the botanical group Alisma, a member of the botanical group Glycyrrhiza and a member of the botanical group Corydalis.

13. The composition of claim 12 further comprising at least one substance selected from the group consisting of beta-carotene, copper, magnesium, rice flour powder, selenium, vitamin B-2, vitamin C, vitamin E, L-carnitine and zinc.

14. The composition of claim 13 comprising danshen, gegen, fuling, baishaoyao, gouqizhi, shanqi, juhua, yanhusuo, mudanpi, shanyao, shanzhuyu, shudihaung, zexie, gancao, huangqi, beta-carotene, copper, magnesium, rice flour powder, selenium, vitamin B-2, vitamin C, vitamin E, L-carnitine and zinc.

15. A method of treating eye discomfort comprising administering to the individual an effective amount of a composition of claim 1.

16. The method of claim 15, wherein the composition comprises fuling, jueminzi, sang ye, shanyao, shanzhuyu, shudihaung, bilberry, danshen, gouqizhi, juhua, baijili, beta-carotene, copper, gujincao, magnesium, mudanpi, rice flour powder, selenium, vitamin B-2, vitamin C, vitamin E, zexie and zinc.

17. The method of claim 15, wherein said eye discomfort is a condition selected from the group consisting of dry eye syndrome, itchy and red eye condition, eye fatigue, and eye sandiness and soreness.

18. A method of treating an eye disease comprising administering to an individual an effective amount of a composition of claim 9.

19. The method of claim 18, wherein said eye disease is age-related macular degeneration and the composition comprises danshen, chongweizi, fuling, zhushizi, gouqizhi, shanqi, juhua, mudanpi, shanyao, shanzhuyu, shudihaung, zexie, gancao, yanhusuo, beta-carotene, copper, magnesium, rice flour powder, selenium, vitamin B-2, vitamin C, vitamin E, L-carnitine and zinc.

20. A method of treating an eye disease comprising administering to an individual an effective amount of a composition of claim 12.

21. The method of claim 20, wherein said eye disease is diabetic retinopathy and the composition comprises danshen, gegen, fuling, baishaoyao, gouqizhi, shanqi, juhua, yanhusuo, mudanpi, shanyao, shanzhuyu, shudihaung, zexie, gancao, huangqi, beta-carotene, copper, magnesium, rice flour powder, selenium, vitamin B-2, vitamin C, vitamin E, L-carnitine and zinc.

22. A method of making a composition for treating eye discomfort, said method comprising combining at least three substances selected from the group consisting of a member of the botanical group poria cocos, a member of the botanical group Cassia, a member of the botanical group Morus, a member of the botanical group Dioscorea, a member of the botanical group officinale and a member of the botanical group Rehmannia.

23. The method of claim 22, wherein said at least three substances are combined with at least one substance selected from the group consisting of a member of the botanical group Vaccinium, a member of the botanical group Salvia, a member of the botanical group Lycium, a member of the botanical group Chrysanthemum, a member of the botanical group Tribulus, beta-carotene, copper, a member of the botanical group Eriocaulon, magnesium, a member of the botanical group Paeonia suffruticosa, rice flour powder, selenium, vitamin B-2, vitamin C, vitamin E, a member of the botanical group Alisma and zinc.
